(19) United States
(12) Patent Application Publication
(10) Pub. No.: US 2026/0202427 A1
(43) Pub. Date: Jul. 16, 2026

(54) METHOD OF PROVIDING EARLY INTERVENTION TO A NEWBORN OR INFANT WITH AUTISM SPECTRUM DISORDER

(71) Applicants: The Trustees of Columbia University in the City of New York, New York, NY (US); The Regents of the University of California, Oakland, CA (US)

(72) Inventors: Xiaoyu CHE, Paramus, NJ (US); Walter Ian LIPKIN, New York, NY (US); Oliver FIEHN, Davis, CA (US); Camilla STOLTENBERG, Oslo (NO)

(21) Appl. No.: 19/387,105

(22) Filed: Nov. 12, 2025

Related U.S. Application Data
(63) Continuation of application No. PCT/US2024/030358, filed on May 21, 2024.
(60) Provisional application No. 63/468,057, filed on May 22, 2023.

(51) Int. Cl. G01N 33/92; G01N 33/68

(52) U.S. Cl. CPC G01N 33/92; G01N 33/6812; G01N 2405/04; G01N 2405/08; G01N 2570/00; G01N 2800/28; G01N 2800/50; G01N 2800/52

(57) ABSTRACT

Described is use of biomarkers found in maternal mid-gestation (MMG) and cord blood (CB) plasma for early identification of children with autism spectrum disorder (ASD) or at risk for ASD. Using these biomarkers, newborns and infants with ASD or at high risk of ASD can be identified and interventions for ASD, which have been shown to be effective early in life, can be provided to a child as young as a newborn.

### CROSS-REFERENCE TO RELATED APPLICATIONS

This application is a continuation of PCT International Application No. PCT/US2024/030358, filed May 21, 2024, which claims benefit of U.S. Provisional Application No. 63/468,057, filed May 22, 2023, the contents of which are hereby incorporated by reference.

### STATEMENT OF GOVERNMENT SUPPORT

This invention was made with government support under NS047537 and NS086122 awarded by the National Institutes of Health. The government has certain rights in the invention.

### BACKGROUND

Approximately 1 in 44 children in the United States have an autism spectrum disorder (ASD) (1). Interventions using applied behavior analysis (ABA) have been proven to be most effective when implemented early (2, 3). However, the mean age for diagnosis is age 4-5 years (4), and the median age varies from 36 to 63 months (1). A recent study has shown that earlier intensive, individualized intervention led to greater improvement in ASD outcomes and further suggested a window of 18 months versus 27 months for the intervention can impact the outcomes. This study shows the need for early identification of children with ASD (124). Accordingly, identification of biomarkers for early diagnosis is a high priority. The pathogenesis of ASD may include genetic, environmental, and epigenetic factors (5).

It is hypothesized that clues to pathogenesis and biomarkers for diagnosis can be revealed through metabolomic analysis of samples from maternal mid-gestation (MMG) and children's cord blood (CB). The few studies of prenatal metabolomic profiling in ASD are inconsistent. One study of MMG sera reported dysregulations in lipid metabolism, pyrimidine metabolism, N-glycan pathway, and C21-steroid hormone biosynthesis (6). Another using maternal sera obtained during the 3rd trimester reported alterations in levels of fatty acid metabolites in the prostaglandin pathway (7). In a third, maternal dyslipidemia and increased levels of serum branched-chain amino acids (BCAA) were associated with an increased risk in boys (8). Metabolomic studies of neonatal samples are typically conducted using blood spots rather than umbilical CB (9, 10). There are no investigations that report metabolomic profiling of both MMG and CB plasma from the same subjects.

Thus, there is a need for markers to make an early diagnosis of ASD in order to provide intervention as early as possible, as early as birth.

### SUMMARY

Described herein are results of metabolomic profiling in MMG and CB plasma drawn from the population-based Norwegian Autism Birth Cohort (ABC) (11). The findings described herein indicate that dysregulated MMG and CB metabolomic profiles are associated with ASD. These dysregulated profiles provide additional evidence for earlier work linking risk to inflammation during gestation as well as new findings consistent with disrupted membrane integrity, impaired neurotransmission, and neurotoxicity. Moreover, the imbalance of ether/non-ether phospholipids in the MMG of ASD girls may provide insight into the higher frequency of cognitive impairment in girls than in boys with ASD.

Disclosed herein are biomarkers that can be found in MMG and CB plasma, for early identification of children at risk for ASD. Using these biomarkers, intervention such as those utilizing ABA, which have been shown to be effective early in life, can be provided to a child as young as a newborn.

One embodiment of the current disclosure is a method for providing early intervention to newborn with autism spectrum disorder (ASD) or at risk for developing ASD comprising the steps of:
identifying that the newborn has ASD or is at risk of developing ASD by:
performing or having performed an ASD association assay on a sample, to identify if the newborn has ASD or is at risk of developing ASD;
identifying the newborn as having ASD or being at risk for developing ASD when there is a dysregulation of the newborn's metabolomic profile consistent with inflammation; and
providing early ASD intervention to the newborn.

In some embodiments, the newborn is a female. In some embodiments, the newborn is a male.

In some embodiments, the sample is blood from the mother at mid-gestation. In some embodiments, the sample is cord blood of the newborn.

In some embodiments, the metabolomic profile is compared to a control metabolomic profile. In some embodiments, the control is a newborn, infant or child not suffering from ASD.

In some embodiments where the newborn is a male and the sample is blood from the mother at mid-gestation, a decreased or lower level of the compound 17-hydroxy-4,7,10,13,15,19-docosahexaenoic acid or chemical cluster hydroxy eicosapentaenoic acid (HEPE) identifies the newborn as having a dysregulated metabolomic profile consistent with inflammation and as having ASD or at risk for developing ASD.

In some embodiments where the newborn is a female and the sample is blood from the mother at mid-gestation, an increased or higher ratio of arachidonic acid (AA)/eicosapentaenoic acid (EPA) identifies the newborn as having a dysregulated metabolomic profile consistent with inflammation and as having ASD or at risk for developing ASD.

In some embodiments where the sample is cord blood, an increased or higher level of the compound AA or ratio of AA/Docosahexaenoic acid (DHA) identifies the newborn as having a dysregulated metabolomic profile consistent with inflammation and as having ASD or at risk for developing ASD, whether the newborn is male or female.

In some embodiments where the newborn is a male and the sample is cord blood, a decreased or lower level of chemical cluster epoxyeicosatrienoic acid (EpETrE) identifies the newborn as having a dysregulated metabolomic profile consistent with inflammation and as having ASD or at risk for developing ASD.

A further embodiment of the current disclosure is a method for providing early intervention to newborn with autism spectrum disorder (ASD) comprising the steps of: identifying that the newborn has ASD or is at risk of developing ASD by:
performing or having performed an ASD association assay on a sample to identify if the newborn has ASD or is at risk of developing ASD; and
identifying the newborn as having ASD or being at risk for developing ASD when there is a dysregulation of the newborn's metabolomic profile consistent with disrupted membrane integrity; and
providing early ASD intervention to the newborn.

In some embodiments, the newborn is a female. In some embodiments, the newborn is a male.

In some embodiments, the sample is blood from the mother at mid-gestation. In some embodiments, the sample is cord blood of the newborn.

In some embodiments, the metabolomic profile is compared to a control metabolomic profile. In some embodiments, the control is a newborn, infant, or child not suffering from ASD.

In some embodiments where the sample is blood from the mother at mid-gestation, a decreased or lower level of chemical cluster phosphatidylcholines (PC), identifies the newborn as having a dysregulated metabolomic profile consistent with disrupted membrane integrity and as having ASD or at risk for developing ASD, whether the newborn is male or female.

In some embodiments where the newborn is a female and the sample is blood from the mother at mid-gestation, a decreased or lower level of compounds PC or phosphatidylethanolamines (PE) or chemical cluster PE, or an increased or higher level of chemical clusters PC-ether or PC ether-vlc identifies the newborn as having a dysregulated metabolomic profile consistent with disrupted membrane integrity and as having ASD or at risk for developing ASD.

In some embodiments where the sample is cord blood, an increased or higher level of chemical cluster ceramide identifies the newborn as having a dysregulated metabolomic profile consistent with disrupted membrane integrity and as having ASD or at risk for developing ASD, whether the newborn is male or female.

In some embodiments where the newborn is a male and the sample is cord blood, a decreased or lower level of chemical cluster PC, or an increased or higher level of chemical clusters carnitine, long chain ceramides or unsaturated ceramides identifies the newborn as having a dysregulated metabolomic profile consistent with disrupted membrane integrity and as having ASD or at risk for developing ASD.

In some embodiments where the newborn is a female and the sample is cord blood, an increased or higher level of chemical cluster sphingomyelin, identifies the newborn as having a dysregulated metabolomic profile consistent with disrupted membrane integrity and as having ASD or at risk for developing ASD.

Yet a further embodiment of the current disclosure is a method for providing early intervention to newborn with autism spectrum disorder (ASD) comprising the steps of:
identifying that the newborn has ASD or is at risk of developing ASD by:
performing or having performed an ASD association assay on a sample to identify if the newborn has ASD or is at risk of developing ASD;
identifying the newborn as having ASD or being at risk for developing ASD when there is a dysregulation of the newborn's metabolomic profile consistent with impaired neurotransmission and neurotoxicity; and
providing early ASD intervention to the newborn.

In some embodiments, the newborn is a female. In some embodiments, the newborn is a male.

In some embodiments, the sample is blood from the mother at mid-gestation. In some embodiments, the sample is cord blood of the newborn.

In some embodiments, the metabolomic profile is compared to a control metabolomic profile. In some embodiments, the control is a newborn, infant or child not suffering from ASD.

In some embodiments where the sample is blood from the mother at mid-gestation, an increased or higher ratio of Glu/Gln, identifies the newborn as having a dysregulated metabolomic profile consistent with impaired neurotransmission and neurotoxicity and as having ASD or at risk for developing ASD, whether the newborn is male or female.

In some embodiments where the newborn is a female and the sample is blood from the mother at mid-gestation, an increased or higher level of compound Glu, identifies the newborn as having a dysregulated metabolomic profile consistent with impaired neurotransmission and neurotoxicity and as having ASD or at risk for developing ASD.

In some embodiments where the newborn is a male and the sample is blood from the mother at mid-gestation, a decreased or lower level of compound Gln, identifies the newborn as having a dysregulated metabolomic profile consistent with impaired neurotransmission and neurotoxicity and as having ASD or at risk for developing ASD.

In some embodiments where the newborn is a male and the sample is cord blood, a decreased or lower level of Gln, or an increased or higher ratio of Glu/Gln, or chemical clusters long chain ceramides or unsaturated ceramides, identifies the newborn as having a dysregulated metabolomic profile consistent with impaired neurotransmission and neurotoxicity and as having ASD or at risk for developing ASD.

Yet a further embodiment of the current disclosure is a method for providing early intervention to newborn with autism spectrum disorder (ASD) comprising the steps of: identifying that the newborn has ASD or is at risk of developing ASD by:
performing or having performed an ASD association assay on a sample to identify if the newborn has ASD or is at risk of developing ASD;
identifying the newborn as having ASD or being at risk for developing ASD when there is a dysregulation of the newborn's metabolomic profile; and
providing early ASD intervention to the newborn.

In some embodiments, the newborn is a female. In some embodiments, the newborn is a male.

In some embodiments, the sample is blood from the mother at mid-gestation. In some embodiments, the sample is cord blood of the newborn.

In some embodiments, the metabolomic profile is compared to a control metabolomic profile. In some embodiments, the control is a newborn, infant or child not suffering from ASD.

In some embodiments where the newborn is a female and the sample is blood from the mother at mid-gestation, an increased or higher level of compounds pyroglutamic acid, propionic acid, N-methylalanine, pseudouridine, PC (p-40:3)/PC (0-40:4), or HexCer-NS, or chemical clusters galactosylceramides or alanine and derivatives, identifies the newborn as having a dysregulated metabolomic profile and as having ASD or at risk for developing ASD. In some embodiments where the newborn is a female and the sample is blood from the mother at mid-gestation, a decreased or lower level of compounds 2,6-dihydroxybenzoic acid, or chemical clusters polyunsaturated fatty acid-containing phosphatidylcholine (PC-PUFA) or hydroxybenzoates, identifies the newborn as having a dysregulated metabolomic profile and as having ASD or at risk for developing ASD.

In some embodiments where the newborn is a male and the sample is blood from the mother at mid-gestation, an increased or higher level of compounds homo-gamma-linolenic acid or oxidized phosphatidylcholine (OxPC), or chemical clusters adipates or unsaturated fatty acids, identifies the newborn as having a dysregulated metabolomic profile consistent and as having ASD or at risk for developing ASD.

In some embodiments where the newborn is a male and the sample is blood from the mother at mid-gestation, a decreased or lower level of chemical clusters hydroxy fatty acid_22_6_1 (OH-FA_22_6_1) or basic amino acids, identifies the newborn as having a dysregulated metabolomic profile consistent and as having ASD or at risk for developing ASD.

In some embodiments where the newborn is a female and the sample is cord blood, an increased or higher level of compounds orotic acid, 2′-O-methylguanosine or eicosatrienoic acid, or chemical cluster purine nucleosides, identifies the newborn as having a dysregulated metabolomic profile and as having ASD or at risk for developing ASD.

In some embodiments where the newborn is a female and the sample is cord blood, a decreased or lower level of chemical clusters triacylglycerols or the majority of unsaturated triglycerides, identifies the newborn as having a dysregulated metabolomic profile and as having ASD or at risk for developing ASD.

In some embodiments where the newborn is a male and the sample is cord blood, an increased or higher level of compounds erythritol, alanine, glucose-6-phosphate, pseudouridine, methionine, succinic acid, 2′-deoxyadenosine-5′-monophosphate, or glycerophosphocholine, or chemical clusters aspartic acids, pyrimidinones, laurates, alanine and derivatives, malates, or sugar alcohols, identifies the newborn as having a dysregulated metabolomic profile consistent and as having ASD or at risk for developing ASD.

In some embodiments where the newborn is a male and the sample is cord blood, a decreased or lower level of compounds 1,4-cyclohexanedione or leukotriene B4, or chemical clusters of nitro compounds, dihydroxyeicosatrienoic acid (DiHETrE), epoxyoctadecadienoic acid (EpODE), PCs, or the majority of hexoses, identifies the newborn as having a dysregulated metabolomic profile consistent and as having ASD or at risk for developing ASD.

In a further embodiment of the current disclosure is a method for providing early intervention to newborn with autism spectrum disorder (ASD) comprising the steps of: identifying that the newborn has ASD or is at risk of developing ASD by:
performing or having performed an ASD association assay on a sample to identify if the newborn has ASD or is at risk of developing ASD;
identifying the newborn as having ASD or being at risk for developing ASD when there is a dysregulation of the newborn's metabolomic profile, wherein the metabolomic profile is determined using one or more of the metabolites listed in Table 7; and
providing early ASD intervention to the newborn.

In some embodiments, the newborn is a female. In some embodiments, the newborn is a male.

In some embodiments, the sample is blood from the mother at mid-gestation. In some embodiments, the sample is cord blood of the newborn.

In some embodiments, the metabolomic profile is compared to a control metabolomic profile. In some embodiments, the control is a newborn, infant or child not suffering from ASD.

In a further embodiment of the current disclosure is a method for providing early intervention to a male newborn with autism spectrum disorder (ASD) comprising the steps of: identifying that the newborn has ASD or is at risk of developing ASD by:
performing or having performed an ASD association assay on a sample to identify if the newborn has ASD or is at risk of developing ASD, wherein the sample is cord blood;
identifying the newborn as having ASD or being at risk for developing ASD when there is high number of dysregulated chemical clusters;
providing early ASD intervention to the newborn.

Lastly a further embodiment of the current disclosure is a method for providing early intervention to a female newborn with autism spectrum disorder (ASD) with increased impaired cognitive development comprising the steps of:
identifying that the newborn has ASD or is at risk of developing ASD with increased impaired cognitive development by:
performing or having performed an ASD association assay on a sample to identify if the newborn has ASD or is at risk of developing ASD with increased impaired cognitive development, wherein the sample is blood from the mother at mid-gestation;
identifying the newborn as having ASD or being at risk for developing ASD with increased impaired cognitive development when there is an imbalance of ether/non-ether phospholipids; and
providing early ASD intervention to the newborn.

As used herein, a control, e.g. a control level or amount or metabolomic profile, is a value or values decided or obtained, usually beforehand, from a sample(s) from a non-afflicted subject(s) as a reference. The concept of a control is well-established in the field, and can be determined, in a non-limiting example, empirically from non-afflicted subjects (versus afflicted subjects, including afflicted subjects having different grades of the relevant affliction), and may be normalized as desired (in non-limiting examples, for volume, mass, age, location, gender) to negate the effect of one or more variables. In embodiments, the control level or amount or metabolomic profile are determined from samples from subjects without ASD.

In all of the foregoing embodiments, an ASD association assay can be performed by any methods known in the art to quantify compounds and/or metabolites from a sample, including a biological sample, including blood.

In all of the foregoing embodiments, the intervention can continue as the newborn ages to an infant and then to a child.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

For the purpose of illustrating the invention, there are depicted in drawings certain embodiments of the invention. However, the invention is not limited to the precise arrangements and instrumentalities of the embodiments depicted in the drawings.

FIGS. 1A-1D. Chemical enrichment analyses reveal sex-specific altered chemical clusters in autism spectrum disorders (ASD). A bar restricted to the left of the centered vertical line indicates a metabolic cluster that is lower in ASD. A bar restricted to the right of the centered vertical line indicates a metabolic cluster that is higher in ASD. A bar that crosses the vertical line indicates a metabolic cluster that is dysregulated in mixed directions. FIG. 1A shows MMG female. FIG. 1B shows MMG male. FIG. 1C shows CB female. FIG. 1D shows CB male.

FIGS. 2A-2D. Autism spectrum disorders (ASD) predictive modeling. FIG. 2A shows ROC curves for MMG girls. FIG. 2B shows ROC curves for MMG boys. FIG. 2C shows ROC curves for CB girls. FIG. 2D shows ROC curves for CB boys. In each of the sex- and sample type-stratified datasets, four machine learning algorithms were used: Lasso; AdaLasso; RF, and XGBoost. Feature selection of metabolites as predictors was conducted using Bayesian analysis and MX knockoffs. For each of the machine learning algorithms, four sets of predictors were considered: Set 1, all metabolites; Set 2, metabolites with BF>3; Set 3, metabolites that were selected by MX knockoffs in more than one iteration; Set 4, metabolites that were selected by MX knockoffs in at least one iteration and had BF>1. The predictive performance was evaluated in 500 iterations of random resampling CV with 80/20 training/testing split.

FIGS. 3A-3C. Compounds, chemical clusters, and metabolite ratios implicated in inflammation (FIG. 3A), membrane integrity (FIG. 3B), and neurotransmission and neurotoxicity (FIG. 3C). Blue arrows indicate elevated, and pink arrows indicate decreased levels of compounds, chemical clusters, and metabolite ratios.

FIG. 4. Pipeline for sample selection. MoBa, the Norwegian Mother, Father, and Child Cohort study, ABC the Norwegian Autism Birth Cohort study.

FIGS. 5A-5B Batch effect correction. FIG. 5A shows PCA scatter plots before correction for batch effect. FIG. 5B shows PCA scatter plots after correction for batch effect. Using scatter plots of the principal components, we detected a batch effect in the complex lipids data from MMG plasma samples that was linked to equipment failure resulting in a one-month delay in completing the lipidomic analyses. Comparisons of variance between batches suggested that mere shifting the means or medians was not sufficient; thus, we implemented a Bayesian framework to correct for the batch effect.

### DETAILED DESCRIPTION

Autism spectrum disorder (ASD) is a neurological and developmental disorder that affects how people interact with others, communicate, learn, and behave. Although autism can be diagnosed at any age, it is described as a “developmental disorder” because symptoms generally appear in the first 2 years of life.

According to the Diagnostic and Statistical Manual of Mental Disorders (DSM-5), a guide created by the American Psychiatric Association that health care providers use to diagnose mental disorders, people with ASD often have difficulty with communication and interaction with other people, restricted interests and repetitive behaviors, and symptoms that affect their ability to function in school, work, and other areas of life.

Autism is known as a “spectrum” disorder because there is wide variation in the type and severity of symptoms people experience.

While much is not known about ASD, what is known is that the earlier intervention is provided to a child with ASD, the better the outcomes. Disclosed herein are methods of identifying newborns with ASD or at risk for developing ASD such that interventions can be started as early as at birth.

Early ASD interventions as contemplated herein are known in the art and include clinician-implemented, and caregiver/patent-implemented. Early ASD interventions can lead to improvements in developmental, adaptive behavior measures, and/or social skills, and include, for example, individual or group Early Social Interaction (ESI) model. In embodiments, by early identification, the methods permit application of early ASD interventions prior to 30 months of age, prior to 20 months of age, or prior to 10 months of age. Early intervention can focus or encompass one or more of: physical skills, thinking skills, communication skills, social skills, and emotional skills. Early intervention can also employ speech therapy, and/or hearing impairment therapy/treatment, and/or physical therapy.

In embodiment, the newborn is male. In embodiment, the newborn is female.

### Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

As used herein, the term “sample” means any substance containing or presumed to contain compounds and/or chemicals and/or metabolites. The sample can be a sample of tissue or fluid isolated from a subject including but not limited to, plasma, serum, whole blood, spinal fluid, semen, amniotic fluid, lymph fluid, synovial fluid, urine, tears, blood cells, organs, and tissue. One sample is blood.

The terms “healthy control” as used herein is a human subject who is not suffering from autism spectrum disorder or at risk for autism spectrum disorder. In addition, a healthy control can be aged matched to the subject being tested, and not suffering from other diseases or conditions.

The term “newborn” as used herein refers to a baby from birth to about two months of age.

The term “infant” as used herein means a baby from about one month to about one year in age.

| Abbreviations | |
|---|---|
| ASD | Autism Spectrum Disorder |
| ID | Intellectual Deficiency |
| ABC | Norwegian Autism Birth Cohort |
| MoBa | Norwegian Mother, Father, and |
| | Child Cohort |
| NPR | Norwegian Patient Register |
| MMG | maternal mid-gestation |
| CB | children's cord blood |
| ABA | applied behavior analysis |
| AdaLasso | adaptive Lasso |
| AUC | area under the curve value |
| BF | Bayesian factor |
| CV | cross-validation |
| Lasso | least absolute shrinkage and |
| | selection operator |
| MX | Model X |
| PCA | principal component analysis |
| RF | Random Forests |
| SD | standard deviation |
| DiHETrE | dihydroxyeicosatrienoic acid |
| EpETrE | epoxyeicosatrienoic acid |
| EpODE | epoxyoctadecadienoic acid |
| HEPE | hydroxy eicosapentaenoic acid |
| OH-FA_22_6_1 | hydroxy fatty acid_22_6_1 |
| PC-ether | ether-linked phosphatidylcholine |
| PC-ether-PUFA | polyunsaturated fatty acid-containing |
| | ether-linked phosphatidylcholine |
| PC-ether-vlc | very long-chain ether-linked |
| | phosphatidylcholine |
| PC-PUFA | polyunsaturated fatty acid-containing |
| | phosphatidylcholine |
| DHA | Docosahexaenoic acid |
| Glu | glutamate |
| Gln | glutamine |
| EPA | Eicosapentaenoic acid |
| AA | arachidonic acid |
| EPA | eicosapentaenoic acid |
| PC | phosphatidylcholine |
| PE | phosphatidylethanolamine |
| CL | complex lipid |
| PM | primary metabolites |
| BA | biogenic amines |
| OL | oxylipins |
| ETrE | eicosatrienoic acid |
| SM | sphingomyelins |
| LPC | saturated lysophosphatidylcholines |

### Tables

### Table 1. Subject Characteristics

^{a}For continuous characteristics, t-test 2-sided p-value; For categorical characteristics, chi-squared 2-sided p-value. ^{b}Parental education missing: MMG, n=35; CB, n=27. ^{c}Folic acid supplements: maternal report of folate exposure between 0-8 weeks of gestation, no missing value. ^{d}Gestational age missing: MMG, n=4; CB, n=3. ªBirth weight missing: MMG, n=2; CB, n=1. ^{f}Intellectual deficiency: We do not have information on ID within controls. ASD autism spectrum disorders, CB cord blood, F female, ID intellectual deficiency, M male, MMG maternal-mid-gestation, N/A not available, SD standard deviation.

| | | MMG Plasma (n = 408) | | | CB Plasma (n = 418) | | |
|---|---|---|---|---|---|---|---|
| | | ASD | Non- | | ASD | Non- | |
| | | Cases | Cases | | Cases | Cases | |
| Subject | | n = 20345 | n = 20547 | p- | n = 20449 | n = 21450 | p- |
| Characteristics | Sex | F/158M | F/158M | value^{a} | F/155M | F/164M | value^{a} |

Maternal Characteristics

| Maternal age; mean | | F | 29.7 | 29.4 | 0.792 | 29.7 | 29.5 | 0.802 |
|---|---|---|---|---|---|---|---|---|
| (SD) | | | (5.4) | (4.1) | | (5.2) | (4.0) | |
| | | M | 29.7 | 30.2 | 0.392 | 29.9 | 30.4 | 0.353 |
| | | | (4.8) | (5.2) | | (4.8) | (4.7) | |
| Parental | <12 | F | 3 | 2 | 0.928 | 4 | 3 | 0.956 |
| education^{b}; | years | | (6.7%) | (4.3%) | | (8.2%) | (6.0%) | |
| n (%) | | M | 9 | 6 | 0.611 | 8 | 6 | 0.199 |
| | | | (5.7%) | (3.8%) | | (5.2%) | (3.7%) | |
| | 12 | F | 7 | 9 | | 7 | 8 | |
| | years | | (15.6%) | (19.1%) | | (14.3%) | (16.0%) | |
| | | M | 47 | 46 | | 46 | 35 | |
| | | | (29.7%) | (29.1%) | | (29.7%) | (21.3%) | |
| | 13-16 | F | 14 | 15 | | 16 | 17 | |
| | years | | (31.1%) | (31.9%) | | (32.7%) | (34.0%) | |
| | | M | 56 | 51 | | 55 | 55 | |
| | | | (35.4%) | (32.3%) | | (35.5%) | (33.5%) | |
| | ≥17 | F | 15 | 18 | | 16 | 19 | |
| | years | | (33.3%) | (38.3%) | | (32.7%) | (38.0%) | |
| | | M | 37 | 46 | | 36 | 52 | |
| | | | (23.4%) | (29.1%) | | (23.2%) | (31.7%) | |

| Folic acid | F | 23 | 30 | 0.217 | 24 | 32 | 0.132 |
|---|---|---|---|---|---|---|---|
| supplements^{c}; n(%) | | (51.1%) | (63.8%) | | (49.0%) | (64.0%) | |
| | M | 85 | 100 | 0.087 | 82 | 104 | 0.057 |
| | | (53.8%) | (63.3%) | | (52.9%) | (63.4%) | |

Child Characteristics

| | | | | | | | | |
|---|---|---|---|---|---|---|---|---|
| Birth year; | 2000 | F | 3 | 0 | 0.142 | 3 | 0 | 0.084 |
| n(%) | | | (6.7%) | (0.0%) | | (6.1%) | (0.0%) | |
| | | M | 4 | 4 | 0.150 | 4 | 3 | 0.010 |
| | | | (2.5%) | (2.5%) | | (2.6%) | (1.8%) | |
| | 2001 | F | 1 | 0 | | 2 | 0 | |
| | | | (2.2%) | (0.0%) | | (4.1%) | (0.0%) | |
| | | M | 12 | 5 | | 10 | 5 | |
| | | | (7.6%) | (3.2%) | | (6.5%) | (3.0%) | |
| | 2002 | F | 7 | 5 | | 7 | 7 | |
| | | | (15.6%) | (10.6%) | | (14.3%) | (14.0%) | |
| | | M | 22 | 10 | | 21 | 8 | |
| | | | (13.9%) | (6.3%) | | (13.5%) | (4.9%) | |
| | 2003 | F | 12 | 6 | | 13 | 6 | |
| | | | (26.7%) | (12.8%) | | (26.5%) | (12.0%) | |
| | | M | 32 | 27 | | 31 | 25 | |
| | | | (20.3%) | (17.1%) | | (20.0%) | (15.2%) | |
| | 2004 | F | 6 | 10 | | 7 | 9 | |
| | | | (13.3%) | (21.3%) | | (14.3%) | (18.0%) | |
| | | M | 26 | 32 | | 23 | 21 | |
| | | | (16.5%) | (20.3%) | | (14.8%) | (12.8%) | |
| | 2005 | F | 8 | 7 | | 8 | 8 | |
| | | | (17.8%) | (14.9%) | | (16.3%) | (16.0%) | |
| | | M | 23 | 25 | | 23 | 21 | |
| | | | (14.6%) | (15.8%) | | (14.8%) | (12.8%) | |
| | 2006 | F | 2 | 5 | | 2 | 6 | |
| | | | (4.4%) | (10.6%) | | (4.1%) | (12.0%) | |
| | | M | 22 | 28 | | 25 | 40 | |
| | | | (13.9%) | (17.7%) | | (16.1%) | (24.4%) | |
| | 2007 | F | 5 | 7 | | 6 | 6 | |
| | | | (11.1%) | (14.9%) | | (12.2%) | (12.0%) | |
| | | M | 12 | 22 | | 13 | 31 | |
| | | | (7.6%) | (13.9%) | | (8.4%) | (18.9%) | |
| | 2008 | F | 1 | 7 | | 1 | 8 | |
| | | | (2.2%) | (14.9%) | | (2.0%) | (16.0%) | |
| | | M | 5 | 4 | | 5 | 8 | |
| | | | (3.2%) | (2.5%) | | (3.2%) | (4.9%) | |
| | 2009 | F | 0 | 0 | | 0 | 0 | |
| | | | (0.0%) | (0.0%) | | (0.0%) | (0.0%) | |
| | | M | 0 | 1 | | 0 | 2 | |
| | | | (0.0%) | (0.6%) | | (0.0%) | (1.2%) | |
| Birth | Winter | F | 11 | 13 | 0.889 | 13 | 13 | 0.825 |
| season; | (December- | | (24.4%) | (27.7%) | | (26.5%) | (26.0%) | |
| n(%) | February) | M | 38 | 47 | 0.049 | 38 | 46 | 0.020 |
| | | | (24.1%) | (29.7%) | | (24.5%) | (28.0%) | |
| | Spring | F | 18 | 18 | | 20 | 22 | |
| | (March- | | (40.0%) | (38.3%) | | (40.8%) | (44.0%) | |
| | May) | M | 33 | 36 | | 34 | 46 | |
| | | | (20.9%) | (22.8%) | | (21.9%) | (28.0%) | |
| | Summer | F | 10 | 8 | | 10 | 7 | |
| | (June- | | (22.2%) | (17.0%) | | (20.4%) | (14.0%) | |
| | August) | M | 31 | 41 | | 31 | 42 | |
| | | | (19.6%) | (25.9%) | | (20.0%) | (25.6%) | |
| | Fall | F | 6 | 8 | | 6 | 8 | |
| | (September- | | (13.3%) | (17.0%) | | (12.2%) | (16.0%) | |
| | November) | M | 56 | 34 | | 52 | 30 | |
| | | | (35.4%) | (21.5%) | | (33.5%) | (18.3%) | |
| Gestational | <37 | F | 3 | 0 | 0.081 | 3 | 0 | 0.185 |
| age^{d}; n(%) | weeks | | (6.7%) | (0.0%) | | (6.1%) | (0.0%) | |
| | | M | 12 | 4 | 0.110 | 12 | 2 | 0.015 |
| | | | (7.6%) | (2.5%) | | (7.7%) | (1.2%) | |
| | 37-42 | F | 42 | 44 | | 45 | 47 | |
| | weeks | | (93.3%) | (93.6%) | | (91.8%) | (94.0%) | |
| | | M | 127 | 137 | | 124 | 144 | |
| | | | (80.4%) | (86.7%) | | (80.0%) | (87.8%) | |
| | ≥42 | F | 0 | 2 | | 1 | 2 | |
| | weeks | | (0.0%) | (4.3%) | | (2.0%) | (4.0%) | |
| | | M | 17 | 16 | | 18 | 17 | |
| | | | (10.8%) | (10.1%) | | (11.6%) | (10.4%) | |
| Birth | <1,500 | F | 0 | 0 | 0.505 | 0 | 0 | 0.459 |
| weight^{e}; | g | | (0.0%) | (0.0%) | | (0.0%) | (0.0%) | |
| n(%) | | M | 3 | 0 | 0.075 | 3 | 0 | 0.077 |
| | | | (1.9%) | (0.0%) | | (1.9%) | (0.0%) | |
| | 1,500- | F | 2 | 0 | | 2 | 0 | |
| | 2,500 g | | (4.4%) | (0.0%) | | (4.1%) | (0.0%) | |
| | | M | 6 | 1 | | 5 | 1 | |
| | | | (3.8%) | (0.6%) | | (3.2%) | (0.6%) | |
| | 2,500- | F | 38 | 40 | | 41 | 41 | |
| | 4,000 g | | (84.4%) | (85.1%) | | (83.7%) | (82.0%) | |
| | | M | 106 | 109 | | 106 | 110 | |
| | | | (67.1%) | (69.0%) | | (68.4%) | (67.1%) | |
| | ≥4,000 | F | 5 | 7 | | 6 | 9 | |
| | g | | (11.1%) | (14.9%) | | (12.2%) | (18.0%) | |
| | | M | 42 | 47 | | 41 | 52 | |
| | | | (26.6%) | (29.7%) | | (26.5%) | (31.7%) | |

| Intellectual | F | 12 | N/A | — | 12 | N/A | F |
|---|---|---|---|---|---|---|---|
| Deficiency^{f}; n(%) | | (26.7%) | | | (24.6%) | | |
| | M | 32 | N/A | — | 29 | N/A | M |
| | | (20.3%) | | | (18.7%) | | |

Table 2. Regression and Bayesian analyses Adjusted logistic regression models with Bayesian inference were used to test for an association between each metabolite and ASD risk, separately for boys and girls and within each sample type. In MMG analyses, models were adjusted for maternal age, illnesses (fever, infection, inflammatory, autoimmune, allergic disorders), emotional distress scores (SCL-5), and non-NSAID antipyretic medications in pregnancy up until sample acquisition, as well as gestational age at MMG blood draw. In CB analyses, models were adjusted for maternal age, illnesses (fever, infection, inflammatory, autoimmune, allergic disorders), emotional distress scores (SCL-5), and non-NSAID antipyretic medications in pregnancy. Multiple comparisons were corrected using the Benjamini-Hochberg procedure, controlling the overall FDR at the level of 0.05. In the Bayesian analysis, we considered a metabolite to be associated with ASD risk if BayesFactor (BF)>10 and 95% HDIs of the estimated aOR did not overlap with 1. Because we used weakly informative priors in Bayesian analysis, the 95% HDIs were similar to the 95% CIs. aOR adjusted odds ratio, ASD autism spectrum disorders, CB cord blood, CI confidence interval, FDR false discovery rate adjusted p-value, HDI highest density credible intervals, MMG maternal mid-gestation.

| Panel | Metabolite | aOR | 95% CI | p-value | FDR | BayesFactor |
|---|---|---|---|---|---|---|
| | MMG Girls | | | | | |
| Biogenic | 2,6- | 0.388 | (0.213, | 0.002 | 0.533 | 39.673 |
| Amines | Dihydroxybenzoic | | 0.707) | | | |
| | acid | | | | | |
| | Glutamic acid - | 2.295 | (1.343, | 0.002 | 0.533 | 34.655 |
| | BA | | 3.921) | | | |
| | Pyroglutamic | 2.121 | (1.265, | 0.004 | 0.533 | 23.775 |
| | acid | | 3.556) | | | |
| | Propionic acid | 2.165 | (1.154, | 0.016 | 0.533 | 21.482 |
| | | | 4.060) | | | |
| | N-Methylalanine | 2.210 | (1.266, | 0.005 | 0.533 | 17.279 |
| | | | 3.855) | | | |
| | Pseudouridine - | 2.105 | (1.236, | 0.006 | 0.533 | 10.659 |
| | BA | | 3.586) | | | |
| Complex | PC (p-40:3)/PC | 2.709 | (1.360, | 0.005 | 0.533 | 118.302 |
| Lipids | (o-40:4) | | 5.399) | | | |
| | TAG (56:9) | 0.452 | (0.235, | 0.017 | 0.533 | 19.200 |
| | | | 0.869) | | | |
| | PE (36:5); PE | 0.424 | (0.246, | 0.002 | 0.533 | 18.499 |
| | (16:0-20:5) | | 0.732) | | | |
| | PC (40:8) - ESI | 0.435 | (0.238, | 0.007 | 0.533 | 17.088 |
| | (+) | | 0.797) | | | |
| | PC (37:5) | 0.454 | (0.254, | 0.008 | 0.533 | 15.595 |
| | | | 0.811) | | | |
| | HexCer-NS | 2.245 | (1.286, | 0.004 | 0.533 | 12.344 |
| | (d34:1) | | 3.921) | | | |
| | MMG Boys | | | | | |
| Complex | FA (20:3) (homo- | 1.548 | (1.229, | 0.000 | 0.251 | 65.460 |
| Lipids | gamma-linolenic | | 1.950) | | | |
| | acid) | | | | | |
| | OxPC (34:2 + 1O); | 1.523 | (1.157, | 0.003 | 0.483 | 18.754 |
| | OxPC (16:0- | | 2.004) | | | |
| | 18:2 + 1O) | | | | | |
| Oxylipins | 17-hydroxy- | 0.667 | (0.523, | 0.001 | 0.483 | 10.980 |
| | 4,7,10,13,15,19- | | 0.850) | | | |
| | docosahexaenoic | | | | | |
| | acid | | | | | |
| | CB Girls | | | | | |
| Primary | Orotic acid - PM | 2.214 | (1.161, | 0.016 | 0.743 | 11.582 |
| Metabolites | | | 4.224) | | | |
| Biogenic | 2′-O- | 2.739 | (1.299, | 0.008 | 0.743 | 23.019 |
| Amines | Methylguanosine | | 5.775) | | | |
| Complex | DAG (36:1) | 2.425 | (1.419, | 0.001 | 0.743 | 69.630 |
| Lipids | | | 4.145) | | | |
| | TAG (56:9) | 0.445 | (0.266, | 0.002 | 0.743 | 19.417 |
| | | | 0.745) | | | |
| | TAG (56:3) | 2.298 | (1.298, | 0.004 | 0.743 | 18.639 |
| | | | 4.068) | | | |
| | TAG (56:8) A | 0.489 | (0.292, | 0.006 | 0.743 | 13.941 |
| | | | 0.818) | | | |
| | TAG (58:10) | 0.472 | (0.281, | 0.004 | 0.743 | 13.223 |
| | | | 0.792) | | | |
| | FA (20:3) | 2.139 | (1.269, | 0.004 | 0.743 | 11.598 |
| | (eicosatrienoic | | 3.605) | | | |
| | acid) | | | | | |
| | CB Boys | | | | | |
| Primary | Erythritol | 1.641 | (1.289, | 0.000 | 0.035 | 242.458 |
| Metabolites | | | 2.089) | | | |
| | Alanine - PM | 1.601 | (1.209, | 0.001 | 0.116 | 26.894 |
| | | | 2.121) | | | |
| | Glucose-6- | 1.504 | (1.184, | 0.001 | 0.116 | 18.599 |
| | phosphate - PM | | 1.911) | | | |
| | Pseudouridine - | 1.804 | (1.226, | 0.003 | 0.138 | 13.556 |
| | PM | | 2.655) | | | |
| | Methionine - PM | 1.489 | (1.172, | 0.001 | 0.116 | 13.064 |
| | | | 1.893) | | | |
| | Succinic acid | 1.452 | (1.147, | 0.002 | 0.116 | 11.444 |
| | | | 1.837) | | | |
| Biogenic | 1,4- | 0.651 | (0.509, | 0.001 | 0.100 | 19.679 |
| Amines | Cyclohexanedione | | 0.834) | | | |
| | 2′- | 1.692 | (1.262, | 0.000 | 0.100 | 18.289 |
| | Deoxyadenosine- | | 2.269) | | | |
| | 5′- | | | | | |
| | monophosphate | | | | | |
| | Glutamine - BA | 0.668 | (0.532, | 0.001 | 0.100 | 17.540 |
| | | | 0.839) | | | |
| | Glycerophosphocholine | 1.460 | (1.153, | 0.002 | 0.102 | 13.239 |
| | | | 1.849) | | | |
| Complex | FA (20:4) | 1.675 | (1.322, | 0.000 | 0.023 | 786.450 |
| Lipids | (arachidonic acid) | | 2.123) | | | |
| Oxylipins | Leukotriene B4 | 0.640 | (0.494, | 0.001 | 0.100 | 24.866 |
| | | | 0.830) | | | |

Table 3. Chemical enrichment analyses

Chemical enrichment analyses of the results from the logistic regression models were performed using ChemRICH to determine chemical clusters that were significantly altered between ASD and control groups. ASD autism spectrum disorders, CB cord blood, DiHETrE dihydroxyeicosatrienoic acid, EpETrE epoxyeicosatrienoic acid, EpODE epoxyoctadecadienoic acid, HEPE hydroxy eicosapentaenoic acid, MMG maternal mid-gestation, OH-FA_22_6_1 hydroxy fatty acid 22_6_1, PC-ether ether-linked phosphatidylcholine, PC-ether-PUFA polyunsaturated fatty acid-containing ether-linked phosphatidylcholine, PC-ether-vlc very long-chain ether-linked phosphatidylcholine, PC-PUFA polyunsaturated fatty acid-containing phosphatidylcholine.

| | | | | | | | |
|---|---|---|---|---|---|---|---|
| | Cluster | p- | Key | | | | Altered |
| Cluster name | size | value | compound | Altered | Increased | Decreased | Ratio |

MMG Girls

| | | | | | | | |
|---|---|---|---|---|---|---|---|
| PC-PUFA | 36 | 4.10E−11 | PC (40:8) - | 12 | 0 | 12 | 0.3 |
| | | | ESI (+) | | | | |
| Galactosylceramides | 6 | 3.10E−08 | GlcCer | 5 | 5 | 0 | 0.8 |
| | | | (d42:2) - ESI | | | | |
| | | | (−) | | | | |
| PC-ether | 23 | 9.80E−07 | PC (o-34:0) | 3 | 3 | 0 | 0.1 |
| PC-ether-vlc | 14 | 2.70E−06 | PC (p- | 6 | 6 | 0 | 0.4 |
| | | | 40:3)/PC (o- | | | | |
| | | | 40:4) | | | | |
| Phosphatidylethanolamines | 44 | 0.0033 | PE (36:5); PE | 5 | 0 | 5 | 0.1 |
| | | | (16:0-20:5) | | | | |
| Phosphatidylcholines | 53 | 0.0072 | PC (37:2) - | 10 | 0 | 10 | 0.2 |
| | | | ESI (+) | | | | |
| PC-ether-PUFA | 22 | 0.013 | PC (p- | 5 | 5 | 0 | 0.2 |
| | | | 42:4)/PC (o- | | | | |
| | | | 42:5) - ESI (−) | | | | |
| Alanine and | 6 | 0.014 | N- | 2 | 2 | 0 | 0.3 |
| derivatives | | | Methylalanine | | | | |
| Hydroxybenzoates | 6 | 0.024 | 2,6- | 2 | 0 | 2 | 0.3 |
| | | | Dihydroxybenzoic | | | | |
| | | | acid | | | | |

MMG Boys

| | | | | | | | |
|---|---|---|---|---|---|---|---|
| 1-Acyl-sn-glycero- | 15 | 2.60E−04 | LPC (18:2) - | 4 | 2 | 2 | 0.3 |
| 3- | | | ESI (+) | | | | |
| phosphocholines | | | | | | | |
| Phosphatidylcholines | 53 | 5.00E−04 | OxPC | 4 | 1 | 3 | 0.08 |
| | | | (34:2 + 1O); | | | | |
| | | | OxPC (16:0- | | | | |
| | | | 18:2 + 1O) | | | | |
| HEPE | 3 | 0.0016 | 12-Hydroxy- | 2 | 0 | 2 | 0.7 |
| | | | 5,8,10,14,17- | | | | |
| | | | eicosapentaenoic | | | | |
| | | | acid | | | | |
| Unsaturated fatty | 26 | 0.005 | FA (20:3) | 4 | 4 | 0 | 0.2 |
| acids | | | (homo-gamma- | | | | |
| | | | linolenic acid) | | | | |
| Saturated | 6 | 0.01 | LPC (14:0) - | 2 | 1 | 1 | 0.3 |
| lysophosphatidylcholines | | | ESI (−) | | | | |
| Basic amino acids | 11 | 0.022 | Asparagine - | 4 | 0 | 4 | 0.4 |
| | | | BA | | | | |
| Adipates | 3 | 0.028 | Adipic acid - | 2 | 2 | 0 | 0.7 |
| | | | BA | | | | |
| OH-FA_22_6_1 | 3 | 0.04 | 17-Hydroxy- | 2 | 0 | 2 | 0.7 |
| | | | 4,7,10,13,15,19- | | | | |
| | | | docosahexaenoic | | | | |
| | | | acid | | | | |

CB Girls

| | | | | | | | |
|---|---|---|---|---|---|---|---|
| Sphingomyelins | 37 | 2.30E−04 | SM (d42:2) B - | 3 | 3 | 0 | 0.08 |
| | | | ESI (−) | | | | |
| Unsaturated | 60 | 0.0012 | TAG (56:3) | 11 | 4 | 7 | 0.2 |
| triglycerides | | | | | | | |
| Purine | 8 | 0.014 | 2′-O- | 2 | 2 | 0 | 0.2 |
| nucleosides | | | Methylguanosine | | | | |
| Ceramides | 6 | 0.023 | Ceramide | 2 | 2 | 0 | 0.3 |
| | | | (d42:2) A - | | | | |
| | | | ESI (+) | | | | |
| Triacylglycerols | 45 | 0.045 | TAG (56:9) | 6 | 0 | 6 | 0.1 |

CB Boys

| | | | | | | | |
|---|---|---|---|---|---|---|---|
| Hexoses | 7 | 5.20E−06 | Mannose - BA | 5 | 1 | 4 | 0.7 |
| Unsaturated fatty | 26 | 6.30E−06 | FA (20:4) | 7 | 5 | 2 | 0.3 |
| acids | | | (arachidonic | | | | |
| | | | acid) | | | | |
| Long-chain | 3 | 6.70E−06 | Ceramide | 3 | 3 | 0 | 1 |
| ceramides | | | (d36:1) - ESI | | | | |
| | | | (+) | | | | |
| DiHETrE | 5 | 1.70E−05 | 19,20- | 3 | 0 | 3 | 0.6 |
| | | | Dihydroxydocosa- | | | | |
| | | | 4,7,10,13,16- | | | | |
| | | | pentaenoic | | | | |
| | | | acid | | | | |
| Carnitines | 9 | 3.70E−05 | Propionylcarnitine | 2 | 2 | 0 | 0.2 |
| Phosphatidylcholines | 53 | 4.70E−05 | PC (32:2) - | 10 | 0 | 10 | 0.2 |
| | | | ESI (−) | | | | |
| Ceramides | 6 | 6.90E−05 | Ceramide | 5 | 5 | 0 | 0.8 |
| | | | (d42:2) A - | | | | |
| | | | ESI (+) | | | | |
| Laurates | 7 | 7.80E−04 | Acylcarnitine | 4 | 4 | 0 | 0.6 |
| | | | (14:1) | | | | |
| Hexosephosphates | 7 | 0.0014 | Glucose-6- | 4 | 3 | 1 | 0.6 |
| | | | phosphate - | | | | |
| | | | PM | | | | |
| EpODE | 7 | 0.0024 | 11-Hydroxy- | 3 | 0 | 3 | 0.4 |
| | | | 14,15- | | | | |
| | | | epoxyeicosatrienoic | | | | |
| | | | acid | | | | |
| EpETrE | 7 | 0.0065 | 12(13)-Epoxy- | 4 | 0 | 4 | 0.6 |
| | | | 9,15- | | | | |
| | | | octadecadienoic | | | | |
| | | | acid | | | | |
| Unsaturated | 16 | 0.0069 | Ceramide | 3 | 3 | 0 | 0.2 |
| ceramides | | | (d36:1) - ESI | | | | |
| | | | (−) | | | | |
| Malates | 4 | 0.01 | Citramalic | 2 | 2 | 0 | 0.5 |
| | | | acid - PM | | | | |
| Sugar alcohols | 14 | 0.018 | Erythritol | 5 | 5 | 0 | 0.4 |
| Pyrimidinones | 5 | 0.028 | Uracil - BA | 3 | 3 | 0 | 0.6 |
| Dipeptides | 26 | 0.03 | Glycyl-leucine | 5 | 4 | 1 | 0.2 |
| Alanine and | 6 | 0.04 | Alanine - PM | 3 | 3 | 0 | 0.5 |
| derivatives | | | | | | | |
| Aspartic acid | 3 | 0.047 | Aspartic acid - | 2 | 2 | 0 | 0.7 |
| | | | PM | | | | |
| Nitro compounds | 3 | 0.049 | 10- | 2 | 0 | 2 | 0.7 |
| | | | Nitrolinoleic | | | | |
| | | | acid | | | | |

### Table 4. Number of Outliers

| | | |
|---|---|---|
| | ASD | Non- |
| Outliers | Cases | Cases |

| | | | |
|---|---|---|---|
| MMG | Primary Metabolites | 2 | 5 |
| Male | Biogenic Amines | 3 | 7 |
| | Complex Lipids | 2 | 4 |
| | Oxylipins | 8 | 5 |
| MMG | Primary Metabolites | 0 | 0 |
| Female | Biogenic Amines | 1 | 0 |
| | Complex Lipids | 0 | 0 |
| | Oxylipins | 3 | 2 |
| CB | Primary Metabolites | 4 | 3 |
| Male | Biogenic Amines | 6 | 9 |
| | Complex Lipids | 3 | 4 |
| | Oxylipins | 1 | 4 |
| CB | Primary Metabolites | 3 | 0 |
| Female | Biogenic Amines | 3 | 1 |
| | Complex Lipids | 1 | 1 |
| | Oxylipins | 3 | 1 |

ASD autism spectrum disorders, CB cord blood, MMG maternal mid-gestation.

Table 5. ASD Associations with Metabolite Ratios

Adjusted logistic regression models were used to test for ASD association with ratios of metabolites. We analyzed ratios of AA/EPA and AA/DHA implicated in inflammation, and ratios of GLU/GLN implicated in neurotransmission. We consider a ratio to be associated with ASD risk (bold) if p-value <0.05. AA arachidonic acid, aOR adjusted odds ratio, ASD autism spectrum disorders, CI confidence interval, DHA docosahexaenoic acid, EPA eicosapentaenoic acid, GLN glutamine, GLU glutamate.

| | | | |
|---|---|---|---|
| Ratio | aOR | 95% CI | p-value |

MMG Girls

| | | | | |
|---|---|---|---|---|
| AA/EPA | 1.893 | 1.133 | 3.162 | 0.015 |
| AA/DHA | 1.552 | 0.936 | 2.573 | 0.088 |
| GLU/GLN | 1.767 | 1.066 | 2.928 | 0.027 |

MMG Boys

| | | | | |
|---|---|---|---|---|
| AA/EPA | 0.916 | 0.725 | 1.157 | 0.461 |
| AA/DHA | 1.028 | 0.817 | 1.292 | 0.815 |
| GLU/GLN | 1.402 | 1.121 | 1.754 | 0.003 |

CB Girls

| | | | | |
|---|---|---|---|---|
| AA/EPA | 1.511 | 0.933 | 2.445 | 0.093 |
| AA/DHA | 1.72 | 1.072 | 2.759 | 0.025 |
| GLU/GLN | 0.888 | 0.569 | 1.385 | 0.6 |

CB Boys

| | | | | |
|---|---|---|---|---|
| AA/EPA | 1.166 | 0.926 | 1.468 | 0.191 |
| AA/DHA | 1.352 | 1.069 | 1.71 | 0.012 |
| GLU/GLN | 1.435 | 1.15 | 1.791 | 0.001 |

ASD autism spectrum disorders, CB cord blood, MMG maternal mid-gestation.

### Table 6. Chemical Enrichment Analyses for Sex x Metabolite Interactions

In MMG and CB analyses, separately, we combined metabolomic data from boys and girls and repeated the adjusted logistic regression models including sex as a covariate, together with the interaction term between sex and each metabolite. ChemRICH analysis was conducted using the estimates of the sex-metabolite interaction terms. CB cord blood, MMG maternal mid-gestation, PC-ether ether-linked phosphatidylcholine, PC-ether-vl c very long-chain ether-linked phosphatidylcholine, PC-PUFA polyunsaturated fatty acid-containing phosphatidylcholine.

| | | | | | | | |
|---|---|---|---|---|---|---|---|
| | | p- | Key | | | | |
| Cluster name | Cluster | valu | compound | Altered | Increased | Decreased | Altered |

MMG

| | | | | | | | |
|---|---|---|---|---|---|---|---|
| PC-PUFA | 36 | 1.90 | PC (40:5) A- | 16 | 16 | 0 | 0.4 |
| Galactosylceramides | 6 | 2.10E−07 | GlcCer | 4 | 0 | 4 | 0.7 |
| | | | (d40:1)- ESI | | | | |
| PC-ether-vlc | 14 | 1.70E−06 | PC (p- | 3 | 0 | 3 | 0.2 |
| | | | 38:4)/PC (o- | | | | |
| PC-ether | 23 | 1.80E−06 | PC (p- | 2 | 0 | 2 | 0.09 |
| | | | 36:4)/PC (o- | | | | |
| Phosphatidylinositols | 15 | 0.0019 | PI (32:0); PI | 2 | 2 | 0 | 0.1 |
| | | | (16:0-16:0) | | | | |
| Phosphatidylethanolamines | 44 | 0.007 | PE (36:5); | 3 | 3 | 0 | 0.07 |
| | | | PE (16:0- | | | | |
| Glycosphingolipids | 5 | 0.01 | HexCer-NS | 2 | 0 | 2 | 0.4 |

CB

| | | | | | | | |
|---|---|---|---|---|---|---|---|
| Hexosephosphate | 7 | 0.00 | Galactosami | 4 | 3 | 1 | 0.6 |
| Cyclohexanes | 6 | 0.017 | 1,4- | 2 | 1 | 1 | 0.3 |
| | | | Cyclohexan | | | | |

### Table 7. Feature Selection Using MX Knockoffs

MX knockoffs teases apart the important variables from noise in high-dimensional datasets while controlling the FDR. We used MX knockoffs with RF, and the procedure was repeated for 500 iterations controlling the FDR at the level of 0.1. The variable importance was measured as the number of iterations in which a metabolomic analyte was selected. CB cord blood, FDR false discovery rate, MMG maternal mid-gestation, MX Model-X, RF-Random Forests.

### Table 7 Feature Selection Using MX Knockoffs.

| MMG Girls | | MMG Boys | |
|---|---|---|---|
| Metabolite | Frequency | Metabolite | Frequency |
| 2,6-Dihydroxybenzcic acid | 88 | OxPC (34:2 + 10); OxPC | 299 |
| | | (16:0-18:2 + 1O) | |
| PE (36:2) - ESI (+) | 56 | FA (20:3) (homo- | 76 |
| | | gamma-linolenic acid) | |
| SM (d36:0) - ESI (+) | 28 | isomaltose A - BA | 64 |
| TAG (50:6) | 20 | Honokiol | 47 |
| PE (36:5); PE (16:0-20:5) | 16 | beta-Hydroxymyristic | 47 |
| | | acid | |
| 14(15)-epoxy-5,8,11,17- | 15 | FA (20:4) (arachidonic | 39 |
| eicosatetraenoic acid | | acid) | |
| SM (d44:2) - ESI (−) | 12 | 5′-Methylthioadenosine | 36 |
| TAG (48:4) A | 11 | PI (36:2); PI (18:0-18:2) | 25 |
| SM (d36:1) - ESI (−) | 10 | Adipic acid - BA | 24 |
| 4-Pyridoxic acid | 8 | Glu-Gln | 17 |
| 5,6-dihydroxyeicosa-8,11,14- | 8 | 1,3 Cyclohexanedione | 7 |
| trienoic acid | | | |
| FA (24:0) (lignoceric acid) | 7 | Glycerophosphocholine | 6 |
| Ceramide (d42:2) A- ESI (−) | 7 | Imatinib | 5 |
| PC (33:2)- ESI (+) | 6 | TAG (50:3) B | 5 |
| PC (34:3) A | 5 | Asparagine - BA | 3 |
| 11,12-Epoxyeicosa-5,8,14- | 5 | FA (24:1) (nervonic acid) | 3 |
| trienoic acid | | | |
| N-Methylhisticine | 4 | N-Methylalanine | 2 |
| PE (40:5); PE (18:0-22:5) | 4 | S-Adenosyl- | 2 |
| | | homocysteine | |
| PC (35:4)- ESI (+) | 4 | Benidipine | 2 |
| 1-Methyl-L-histidine | 3 | CE (16:1) | 2 |
| PC (40:8)- ESI (+) | 3 | glucose-6-phosphate- | 1 |
| | | PM | |
| PC (37:5) | 3 | N-Acetylalanine | 1 |
| PC (37:3) A | 3 | Betaine | 1 |
| PC (32:2)- ESI (+) | 3 | Liquiritigenin | 1 |
| Glutamic acid - BA | 2 | Orotic acid - BA | 1 |
| Ethronolactone | 2 | Pipecolic acid | 1 |
| N-Acetylphenylalanine | 2 | Riboflavin | 1 |
| PC (36:5) D - ESI (+) | 2 | 1,2-Benzenedicarbolxylic | 1 |
| | | acid | |
| PC (34:3) B | 2 | TAG (51:5) | 1 |
| PE (34:2) - ESI (+) | 2 | TAG (60:4) | 1 |
| SM (d42:2) B - ESI (+) | 2 | PC (35:2) - ESI (−) | 1 |
| TAG (50:4) | 2 | SM (d42:2) B - ESI (+) | 1 |
| PC (34:4)- ESI (+) | 2 | FA (22:1) (erucic acid) | 1 |
| PE-Cer (d42:2) | 2 | DAG (34:2) | 1 |
| 1-methylgalactose | 1 | PC (40:4)- ESI (−) | 1 |
| glycerol-alpha-phosphate | 1 | CE (20:2) | 1 |
| isomaltose A - BA | 1 | TAG (53:0) | 1 |
| Glycyl-leucine | 1 | TAG (58:4) | 1 |
| Methylacetate | 1 | | |
| Picrotin | 1 | | |
| Acetaminophen glucuronide | 1 | | |
| Kynurenine - BA | 1 | | |
| N-Acetylcytidine | 1 | | |
| PC (37:3) B | 1 | | |
| PE (p-38:5)/PE | 1 | | |
| (0-38:6) - ESI (−) | | | |
| PC (36:4) B - ESI (+) | 1 | | |
| PE (34:2) - ESI (−) | 1 | | |
| PC (p-38:4)/PC | 1 | | |
| (0-38:5) B - ESI (−) | | | |
| PC (38:7)- ESI (+) | 1 | | |
| FA (22:2) (docosadienoic acid) | 1 | | |
| SM (d42:2) B- ESI (−) | 1 | | |
| Ceramide (d36:1)- ESI (−) | 1 | | |
| PC (p-40:1)/PC | 1 | | |
| (0-40:2)- ESI (+) | | | |
| 14-hydroxydocosa-4,7,10,11, | 1 | | |
| 16,19-hexaenoic acid | | | |
| 5-Hydroxy-6,8,11,14- | 1 | | |
| eicosatetraenoic acid | | | |

### Table 8. AUC Values of Predictive Models

In each of the sex- and sample type-stratified datasets, we employed four machine learning algorithms: Lasso, AdaLasso, RF, and XGBoost. Feature selection of metabolites as predictors was conducted using Bayesian analysis and MX knockoffs. For each of the machine learning algorithms, we considered four sets of predictors: Set 1, all metabolites; Set 2, metabolites with BF>3; Set 3, metabolites that were selected by MX knockoffs in more than one iteration; Set 4, metabolites that were selected by MX knockoffs in at least one iteration and had BF>1. The predictive performance was evaluated in 500 iterations of random resampling CV with 80/20 training/testing split. AdaLasso adaptive Lasso, AUC area under the curve value, CB cord blood, CI confidence interval, CV cross-validation, Lasso least absolute shrinkage and selection operator, MMG maternal mid-gestation, RF Random Forests.

| CB Girls | | CB Boys | |
|---|---|---|---|
| Metabolite | Frequency | Metabolite | Frequency |
| PC (34:0)- ESI (−) | 55 | erythritol | 211 |
| PE (34:3e); PE | 38 | Galactceamine | 95 |
| PE (p-34:2)/PE | 31 | FA (20:4) | 87 |
| (o-34:3) | | (arachidonic acid) | |
| 2′-O- | 28 | N-acetylornithine | 67 |
| Methylguanosine | | | |
| DAG (36:1) | 19 | 9-ketooctadeca-10,12- | 57 |
| | | dienoic acid | |
| Ala-Ala | 18 | FA (20:3) | 56 |
| | | (eicosatrienoic acid) | |
| PE (p-36:2)/PE | 16 | PC (p-34:2)/PC | 55 |
| (o-36:3) - ESI (+) | | (o-34:3)- ESI (−) | |
| 8-Oxo-2- | 9 | TAG (60:3) | 55 |
| deoxyadenoeine | | | |
| Phosphomycin | 9 | Glutamine - BA | 53 |
| Acylcarnitine (12:1) | 9 | PC (33:2)- ESI (−) | 53 |
| Ribose B - BA | 8 | hypoxanthine - PM | 25 |
| Nateglinide | 8 | inosine - PM | 24 |
| LPE (20:4) - ESI (+) | 8 | Mannose - BA | 22 |
| Formoterol | 7 | Ceramide (d36:1)- ESI (+) | 19 |
| pseudouridine- PM | 6 | TAG (64:2) | 17 |
| DAG (36:2) | 6 | 12,13-dihydroxyoctadeca-9, | 17 |
| | | 15-dienoic acid | |
| Glucuronic acid A- | 4 | Ferulic acid | 16 |
| BA | | | |
| TAG (56:3) | 4 | Ceramide (d40:2) | 16 |
| 2-deoxytetronic acid | 3 | 3-(1-Pyrazolyl)-alanine | 10 |
| PC (p-42:5)/PC | 3 | Cyclo(Leu-Pro) | 10 |
| (o-42:6)- ESI (−) | | | |
| SM (d42:2) B- | 3 | 2′-Deoxyadenosine- | 9 |
| ESI (−) | | 5′-monophosphate | |
| PC (44:6e) | 3 | Leukotriene B4 | 9 |
| glycerol | 2 | N-Methylalanine | 8 |
| Kaempferol-3-0- | 2 | 3-Hydroxyphenylacetic acid | 7 |
| rutinoside | | | |
| N-Acetylcytidine | 2 | 11,12-Dihydroxyicosa-5,8, | 7 |
| | | 14-trienoic acid | |
| PI (32:1); PI | 2 | oxoproline | 6 |
| (16:0-16:1) | | | |
| PC (36:3) C- ESI (+) | 2 | Histicine- BA | 6 |
| TAG (56:2) | 2 | Acylcarnitine (10:0) | 6 |
| PI (34:1); PI | 2 | Hypoxanthine - BA | 5 |
| (16:0- 18:1) | | | |
| PC (p-36:1)/PC | 2 | 3′-O-Methylguanosine | 5 |
| (o-36:2)- ESI (-) | | | |
| PC (38:6) A- ES! (+) | 2 | Ceramide (d42:2) A- ESI (+) | 5 |
| PI (36:1); PI | 2 | 14, 15-dihydroxyeicosa-5,8, | 5 |
| (18:0-18:1) | | 11-trienoic acid | |
| 6-trans-Leukotriene | 2 | 8-Oxo-2-deoxyadenoeine | 4 |
| B4 | | | |
| Carboxyibuprofen | 1 | N-.alpha.-(tert-Butoxycartonyl)-L- | 4 |
| 2,6- | 1 | 18-(3-ethyloxiran-2-yl)octadeca- | 4 |
| Dihydroxybenzoic | | 4,7,10,13,16-pentaenoic acid | |
| acid | | | |
| 1,4- | 1 | 14-hydroxydocosa-4,7,10,12, | 4 |
| Cyclohexane- | | 16,19-hexaenoic acid | |
| dicarboxylic acid | | | |
| Piroxicam | 1 | pseudouridine- PM | 3 |
| 3-Indoleprodionic | 1 | ribitol | 3 |
| acid | | | |
| Methylgallate | 1 | glycerol-alpha-phosphate | 3 |
| Ergothioneine | 1 | 1,4-Cyclohexanedione | 3 |
| Glucose-1-phosphate | 1 | Citramalic acid- BA | 3 |
| Cortodoxone | 1 | Fenfluramine B | 3 |
| Thr-Glu | 1 | PI (37:4) | 3 |
| Tomelukast | 1 | xanthine | 2 |
| TAG (56:9) | 1 | 2-Hydroxyphenylacetic acid | 2 |
| TAG (54:8) | 1 | Benzphetamine | 2 |
| TAG (54:9) | 1 | Inosine - BA | 2 |
| PC (40:6) A- ESI (−) | 1 | PC 34:3 | 2 |
| PC (p-34:1)/PC | 1 | TAG (54:8) | 2 |
| (o-34:2)- ESI (+) | | | |
| PE (p-38:5)/PE | 1 | 12(13)-epoxy-9,15- | 2 |
| (o-38:6) - ESI (+) | | octadecadienoic acid | |
| PC (0-34:0) | 1 | 12-Hydroxy-5,8,10, | 2 |
| | | 14-eicosatetraenoic acid | |
| LPE (22:6) | 1 | aspartic acid - PM | 1 |
| SM (d44:2) - ESI (−) | 1 | sucrose | 1 |
| PC (34:3) B | 1 | glycerol | 1 |
| | | adipic acid - PM | 1 |
| | | cerotinic acid | 1 |
| | | Glycerophosphocholine | 1 |
| | | Tryptophan - BA | 1 |
| | | Uracil- BA | 1 |
| | | Glycyl-leucine | 1 |
| | | Coniferylaldehyde | 1 |
| | | Inosine-5-monophosphate | 1 |
| | | Docosahexanoic acid | 1 |
| | | Gly-val | 1 |
| | | Orotic acid - BA | 1 |
| | | N-Acetylglycine | 1 |
| | | 3-Methyladipic acid | 1 |
| | | Ceramide (d34:1)-ESI (+) | 1 |
| | | PC (32:2)-ESI (−) | 1 |
| | | PC (35:2)- ESI (−) | 1 |
| | | PC (36:5) A- ESI (+) | 1 |
| | | FA (14:1) (physeteric acid) | 1 |
| | | PE (36:2) - ESI (+) | 1 |
| | | 15(16)-epoxy-9,12- | 1 |
| | | octadecadienoic acid | |
| | | Thromboxane B2 | 1 |
| | | Leukotriene B5 | 1 |

MX knockoffs teases apart the important variables from noise in high-dimensional datasets while controlling the FDR. We used MX knockoffs with RF, and the procedure was repeated for 500 iterations controlling the FDR at the level of 0.1. The variable importance was measured as the number of iterations in which a metabolomic analyte was selected. CB cord blood, FDR false discovery rate, MMG maternal mid-gestation, MX Model-X, RF Random Forests.

### Markers of Autism Spectrum Disorder (ASD)

Described herein are results of metabolomic profiling in MMG and CB plasma drawn from the population-based Norwegian Autism Birth Cohort (ABC) (11). The findings described herein indicate that dysregulated MMG and CB metabolomic profiles are associated with ASD. These dysregulated profiles provide additional evidence for earlier work linking risk to inflammation during gestation as well as new findings consistent with disrupted membrane integrity, impaired neurotransmission, and neurotoxicity. Moreover, the imbalance of ether/non-ether phospholipids in the MMG of ASD girls may provide insight into the higher frequency of cognitive impairment in girls than in boys with ASD.

The discovery of prenatal and neonatal molecular biomarkers has the potential to yield insights into autism spectrum disorder (ASD) and facilitate early diagnosis. Metabolomic profiles in ASD were analyzed using plasma samples collected in the Norwegian Autism Birth Cohort from mothers at weeks 17-21 gestation (maternal mid-gestation, MMG, n=408) and from children on the day of birth (cord blood, CB, n=418). Associations were analyzed using sex-stratified adjusted logistic regression models with Bayesian analyses. Chemical enrichment analyses (ChemRICH) were performed to determine altered chemical clusters. Machine learning algorithms were also used to assess the utility of metabolomics as ASD biomarkers.

ASD was associated with a variety of chemical compounds including arachidonic acid, glutamate, and glutamine, and metabolite clusters including hydroxy eicospentaenoic acids, phosphatidylcholines, and ceramides in MMG and CB plasma that are consistent with inflammation, disruption of membrane integrity, and impaired neurotransmission and neurotoxicity. Girls with ASD have disruption of ether/non-ether phospholipid balance in the MMG plasma that is similar to that found in other neurodevelopmental disorders. ASD boys in the CB analyses had the highest number of dysregulated chemical clusters. These findings may provide new insights into the sex-specific differences in ASD and have implications for discovery of biomarkers that may enable early detection and intervention.

Specifically, the following dysregulation of compounds, ratios and chemical clusters were found in the MMG and CB of subjects with ASD in the study described herein:

### Compounds and Ratios

Females-MMG
increased or higher levels of compounds: glutamic acid; pyroglutamic acid; propionic acid; N-methylalanine; pseudouridine; PC (p-40:3)/PC (0-40:4), HexCer-NS; AA/EPA, and Glu/Gln
decreased or lower levels of compounds: 2,6-dihydroxybenzoic acid;
triglyceride (TAG); PC; and PE/PE

Females-CB
increased or higher levels of compounds: orotic acid; 2′-O-methylguanosine; EtrE; TAG; and diglyceride (DAG); and ratios of AA/DHA decreased or lower levels of TAGs

Males-MMG
increased or higher levels of compounds homo-gamma-linolenic acid; and OxPC; and ratios of Glu/Gln
decreased or lower levels of 17-hydroxy-4,7,10,13,15,19-docosahexaenoic acid

Males-CB
increased or higher levels of compounds: AA; erythritol; alanine; glucose-6-phosphate; pseudouridine; methionine; succinic acid; 2′-deoxyadenosine-5′-monophosphate; and glycerophosphocholine; and ratios of AA/DHA; and Glu/Gln
decreased or lower levels of compounds glutamine, 1,4-cyclohexanedione and leukotriene B4

### Chemical Clusters

Females-MMG
increased or higher levels of galactosylceramides, very long-chain ether-linked phosphatidylcholine (PC-ether-vlc), alanine and derivatives, polyunsaturated fatty acid-containing ether-linked phosphatidylcholine (PC-ether-PUFA), and ether-linked phosphatidylcholine (PC-ether)
decreased or lower levels of polyunsaturated fatty acid-containing phosphatidylcholine (PC-PUFA), hydroxybenzoates, phosphatidylcholines (PC), and phosphatidylethanolamines (PE)

Females-CB
increased or higher levels of ceramides, purine nucleosides, and sphingomyelins (SM)
decreased or lower levels of triacylglycerols and the majority of unsaturated triglycerides

Males-MMG
increased or higher levels of adipates and unsaturated fatty acids
decreased or lower levels of hydroxy eicosapentaenoic acid (HEPE), hydroxy fatty acid_22_6_1 (OH-FA_22_6_1), basic amino acids, and the majority of phosphatidylcholines (PC) mixed direction levels of 1-acyl-sn-glycero-3-phosphocholines, and saturated lysophosphatidylcholines (LPC)

Males-CB
increased or higher levels of long-chain ceramides, ceramides, aspartic acids, pyrimidinones, laurates, alanine and derivatives, malates, sugar alcohols, unsaturated ceramides, carnitines, and the majority of dipeptides, unsaturated fatty acids, and hexosephosphates
decreased or lower levels of nitro compounds, dihydroxyeicosatrienoic acid (DiHETrE), epoxyeicosatrienoic acid (EpETrE), epoxyoctadecadienoic acid (EpODE), PCs, and the majority of hexoses

Additionally, certain chemical clusters were found that were significantly different with regard to sex. In MMG, girls with ASD had lower levels of PC-PUFA, galactosylceramides, PC-ether-vlc, PC-ether, phosphatidylinositols (PI), PEs, and glycosphingolipids with no differences in boys. In CB, two chemical clusters with sex differences in ASD association: hexosephosphates and cyclohexanes. The ASD associations with these metabolites were in opposite directions between boys and girls (increased in ASD boys but decreased in ASD girls; or decreased in ASD boys but increased in ASD girls).

MMG was a focus based on studies that indicate the early gestational environment is an important factor in ASD outcome: Swedish registry studies wherein first trimester use of the antiseizure medication valproic acid was associated with ASD risk (44), increased risk with thalidomide exposure at 20-24 days gestation (45) and earlier work in the ABC showing a robust protective effect of dietary folate in women who initiated supplementation before conception or during early gestation (43). Further support came from rodent models for ASD based on dam early-midgestational exposure to infection, stimulants of innate immunity or toxins (46, 47, 48).

The majority of papers describing metabolomic profiles in maternal plasma represent results from high-risk cohorts where the pathogenesis of ASD may be skewed towards specific genetic mechanisms (7, 49, 50, 51, 52) or environmental exposures (53). An exception is a population-based study in California where findings included dysregulations in lipid metabolism, pyrimidine metabolism, N-glycan pathway, and C21-steroid hormone biosynthesis (6).

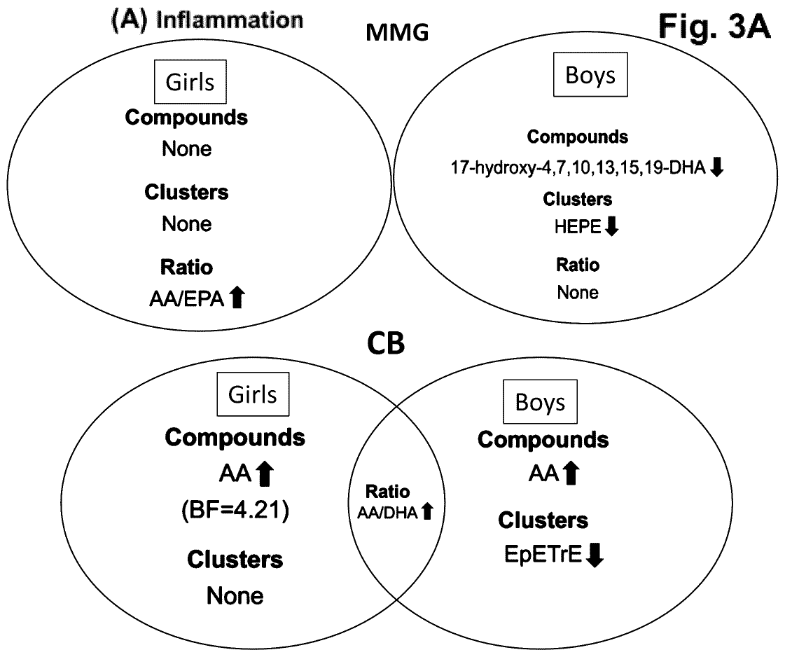

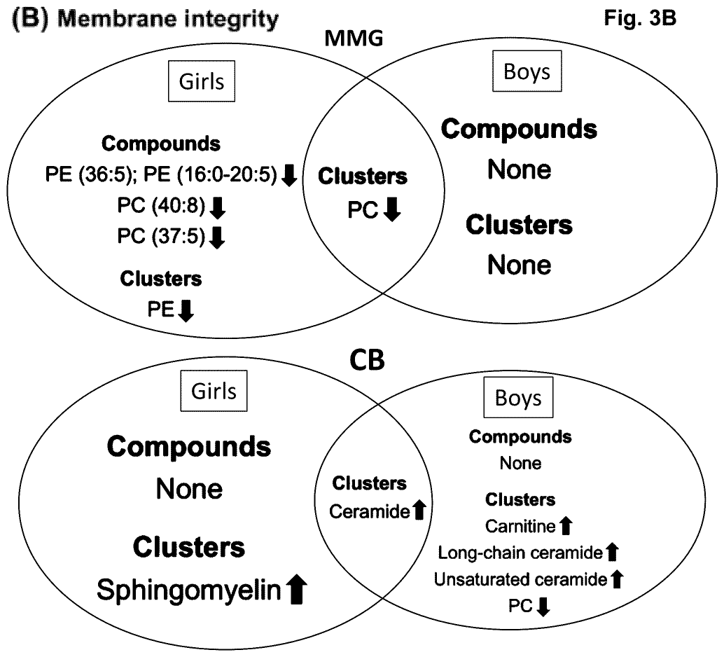

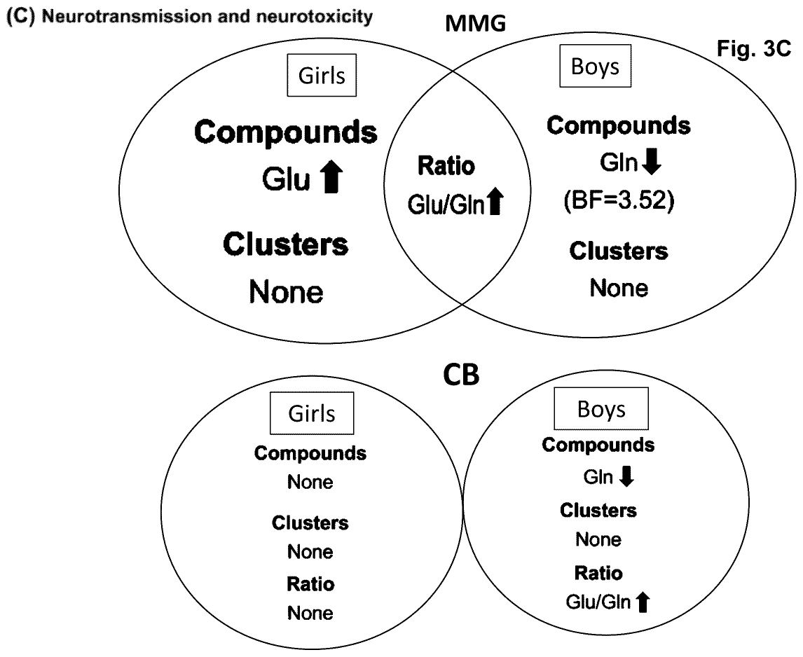

The key findings shown herein in boys and girls with ASD include dysregulated MMG and CB metabolomic profiles consistent with inflammation (FIG. 3A), disruption of membrane integrity (FIG. 3B), and impaired neurotransmission and neurotoxicity (FIG. 3C). Girls with ASD also have disruption of ether/non-ether phospholipid balance in the MMG that is reminiscent of other neurodevelopmental disorders (54, 55, 56). The sample size was smaller for girls than boys; however, subsampling and post hoc power analyses suggested that the differences in findings between boys and girls were not due to differences in sample size.

### Inflammation

A recent literature review of publications reporting analyses of more than 411 ASD subjects and 596 healthy controls through post-mortem histology and in vivo positron emission tomography revealed microglial activation in ASD (60). RNASeq analyses of human cortex from 47 ASD cases and 57 controls found evidence of microglial activation and type 1 interferon responses (61).

In MMG studies reported herein, analytes in the HEPE chemical cluster were decreased in ASD boys, but not in girls. ASD boys also had decreased levels of 17-hydroxy-4,7,10,13,15,19-docosahexaenoic acid compared to controls. HEPE and DHA are omega-3 (n-3) polyunsaturated fatty acids (PUFA) that are precursors to anti-inflammatory leukotrienes, resolvins and protectins and contribute to resolution of inflammation (62). Supplementation of HEPEs and DHAs reduce TNF-α expression (63). Studies in animal models have revealed that HEPEs regulate inflammation by induction of regulatory T cells (Tregs) (64), and that DHAs inhibit LPS-induced TNF-α production in macrophages (65).

In CB, AA was elevated in ASD boys, but not in girls. AA was also elevated (BF=4.21) in ASD boys in MMG but did not meet the criterion for BF>10. AAs are omega-6 (n-6) PUF As that are metabolized by cyclooxygenases (COX) and lipoxygenases (LOX) to produce pro-inflammatory eicosanoids (66). In human cell lines, AAs stimulate the c-jun amino-terminal kinase (JNK) cascade and NF-κB signaling (67), and promote TNF-α production (68). Levels of analytes in the EpETrE chemical cluster were reduced in ASD boys, but not in girls. EpETrEs are anti-inflammatory mediators derived from AAs through the action of cytochrome P450 (CYP450) epoxygenases (69). EpETrEs inhibit NF-κB signaling, prevent the accumulation of NF-κB subunit Rel A, and regulate TNF-α-induced inflammation in human endothelial cells (69).

A previous study reported imbalance in the n-6 PUFA/n-3 PUFA ratio in plasma of ASD subjects that is consistent with inflammation (39). Accordingly, ratios of AA/DHA and AA/EPA between ASD and controls were compared in each of the sex- and sample type-stratified datasets (Table 5). In MMG, AA/EPA ratios were elevated in ASD girls, but not in boys. Both boys and girls with ASD had elevated AA/DHA ratios in CB. Elevations in AA/EPA ratios are consistent with inflammation. Elevations in AA/DHA ratios are implicated in reactive oxygen species (ROS)-induced inflammation in hepatoma cell lines (70). Reductions in AA/DHA ratios inhibit TNF-α-induced inflammation in alveolar cells (71). In a recent plasma cytokine profile analysis with the same cohort reported here (72), we found prominent TNF-α elevations in the MMG and CB of both ASD boys and girls.

The metabolomic analyses set forth herein provide evidence of dysregulation in fatty acid metabolism that is consistent with lipid-mediated gestational inflammation.

### Disrupted Membrane Integrity

Reduced levels of analytes in the PC chemical cluster were found in MMG in both ASD girls and boys, and in CB of ASD boys, but not girls. PCs are abundant in the membranes of cells and organelles, and regulate membrane integrity, transport, and G protein-coupled receptor (GPCR) signaling (73, 74). PC depletion affects the stability of the membrane protein translocases, impairs membrane integrity, and induces apoptosis (75, 76). In MMG, levels of analytes in the PE chemical cluster were reduced in ASD girls, but not in boys. PEs are integral membrane components essential for membrane fusion and stability (74). PE depletion disrupts membrane dynamics with altered membrane transport and receptor signaling (77), and impairs autophagy (78). In CB, we found elevated levels of analytes in the carnitine chemical cluster in ASD boys, but not in girls. Carnitines facilitate the transport of fatty acids across mitochondrial membranes (79). Previous studies reported elevations of short chain acyl-carnitines in plasma of ASD human subjects (80) and in brain tissues of ASD rodents (81) that are consistent with impaired membrane transport in ASD.

Levels of analytes in the ceramide chemical cluster were elevated in CB of both ASD boys and girls. The levels of metabolites in the long-chain ceramide and unsaturated ceramide chemical clusters were increased in CB of ASD boys, but not in girls. Ceramides are bioactive sphingolipids of biological membranes that regulate membrane packing and membrane fusion (82). Apart from controlling cellular signaling, these sphingolipids control cell fate (83). Ceramide accrual has been implicated in gestational complications including preeclampsia (84) and neural tube defects (85). As key components of the neuronal membranes, ceramides are critical to neuronal health (86). These sphingolipids induce neuronal apoptosis (87), and promote neurodegeneration (88). Levels of metabolites in the SM chemical cluster were elevated in the CB of ASD girls, but not in boys. SMs are phosphocholine derivatives of ceramides (89) that are integral components of membrane lipid microdomains or “lipid rafts” (90). SMs regulate exocytotic processes and membrane fusion (90). Accumulation of SMs disrupt maturation and closure of autophagic membranes and leads to impaired autophagy in Niemann-Pick type A (NPA) disease (91).

Intake of selective SSRIs is reported to affect levels of plasma lipid profiles (92, 93). Only a small number of ABC subjects received SSRIs during pregnancy (n=1 in MMG girls, n=7 in MMG boys, n=1 in CB girls, n=8 in CB boys). Repeated analyses excluding these subjects had no impact of group-specific chemical clustering (data not shown).

Significant sex differences in ASD associations with lipid chemical clusters in MMG were found. Whereas ASD versus control girls had lower levels of analytes in the PC-PUFA, PI, and PE chemical clusters, and higher levels of analytes in the PC-ether and PC-ether-vlc chemical clusters, ASD boys had no alterations in these clusters. PEs, PIs and PC—PUFAs are non-ether phospholipids, whereas PC-ethers and PC-ether-vlcs are ether phospholipids of peroxisomal origin (94). Ether and non-ether phospholipids are critical to membrane integrity and dynamics (74, 94). As key components of neuronal membranes, they regulate function (94). ASD girls exhibit lower cognitive ability than ASD boys (95). Elevations in ether phospholipids with concomitant reductions in non-ether phospholipids in the MMG plasma of ASD girls may indicate lipid remodeling consistent with a shift in the plasma lipidome toward ether phospholipids from the non-ether equivalents. An ether/non-ether phospholipid imbalance, with a shift toward ether phospholipids, is reported with de novo variations in fatty acyl-CoA reductase 1 (FAR1), Sjögren-Larsson syndrome, and complex hereditary spastic paraplegia due to phosphate cytidylyltransferase 2, ethanolamine (PCYT2) mutations (54, 55, 56).

We obtained information on the cognitive dysfunctions as reflected in the diagnosis of intellectual deficiency (ID) in ASD cases. Compared to ASD girls without ID, ASD girls with ID had decreased levels in MMG plasma of analytes in the PE cluster (altered ratio 0.16, 100% decreased, p-value <0.0001), and increased levels of analytes in basic amino acid cluster (altered ratio 0.36, 75% increased, p-value <0.001).

### Impaired Neurotransmission and Neurotoxicity

In MMG, ASD girls, but not boys, had elevations in the levels of Glu. Levels of Gln were reduced in ASD boys (BF=3.52), but not in girls, though it did not meet the criterion for BF>10. In CB, ASD boys, but not girls, had reduced levels of Gln. Dysregulations in Glu/Gln metabolism with elevated plasma Glu and reduced plasma Gln are reported in children with ASD (96, 97). However, to our knowledge, this is the first evidence of dysregulations in Glu/Gln metabolism during gestation. Glu is an excitatory neurotransmitter that is critical in neuronal migration and plasticity, and synaptogenesis (98). Nonetheless, excess Glu leads to dysfunction of glutamatergic neurotransmission (98), and promotes excitotoxicity-induced neuronal apoptosis (99, 100). In a recent plasma cytokine profile analysis with the same cohort reported here, we found prominent TNF-α elevations in MMG plasma of ASD subjects (72). TNF-α stimulates Glu production and promotes excitoneurotoxicity in murine microglial cells (101). TNF-α also increases blood-brain barrier permeability in animal models (102). Although Glu does not readily cross an intact blood-brain barrier (103), the observation that TNF-α is elevated may explain the correlation between elevated levels of Glu in plasma and in brain in ASD (104).

Gin is a precursor to both excitatory (Glu, aspartate) and inhibitory (y-aminobutyric acid) neurotransmitters (105). Gln also has a neuroprotective role (106, 107). Given studies reporting elevated plasma Glu/Gln ratios in ASD (40, 41), we compared Glu/Gln ratios between ASDs and controls in each of the sex- and sample type-stratified dataset (Table 5). Glu/Gln ratios were elevated in both ASD boys and girls in MMG plasma. In the CB plasma, ASD boys, but not ASD girls, had elevated Glu/Gin ratios.

We previously reported that prenatal folic acid supplementation is associated with decreased risk of ASD (43). Folic acid has also been shown to reduce glutamate-induced excitotoxicity (108). Accordingly, we tested whether folic acid supplementation confounded results. The elevations of Glu/Gln ratios in ASD were not confounded by maternal intake of folic acid supplements (data not shown).

The analyses disclosed herein provide evidence for dysregulations in the Glu/Gln metabolism in gestation that are consistent with the neuropathology of ASD.

### Testing for Biomarkers

As shown herein, certain metabolite and chemical markers and chemical clusters and ratios are associated with ASD. By using these markers, important predictions and determinations can be made regarding a newborn. Tests for these biomarkers can be performed at birth or during pregnancy. Blood or plasma can be obtained from cord blood at birth or from a routine pregnancy checkup of the mother.

The presence or amount of the markers can be compared to a reference value. In some embodiments, the reference value is from a healthy control, i.e., a newborn, infant or child not suffering from an ASD. In some embodiments, the reference value is from a newborn, infant or child with an ASD.

In certain embodiments, a sample of fluid from a subject is obtained. In some embodiments, the subject is a newborn. In some embodiments, the subject is a pregnant mother. In some embodiments, the fluid is blood. In some embodiments, the fluid is plasma.

Any method known in the art can be used to test for the biomarkers in the sample, including but not limited to targeted bioactive oxylipin assay, and chromatography/mass spectrometry-based assays (MS) including but not limited to gas chromatography/time-of-flight mass spectrometry (GC-TOF MS), hydrophilic interaction liquid chromatography/quadrupole time-of-flight mass spectrometry (HILIC-QTOF MS), and liquid chromatography (LC)/quadrupole time-of-flight mass spectrometry (CSH-QTOF MS).

### Early Intervention for Autism Spectrum Disorders

One major advantage of the present disclosure is it allows the identification or diagnosis of newborns who have or will develop or are at high risk of developing an ASD. This allows intervention at the earliest possible stages of development, which increases the success of the intervention.

Using the biomarkers disclosed herein, if a newborn is identified or diagnosed with an ASD, interventions can be provided to the newborn or to the newborn as they become an infant and beyond including but not limited to applied behavioral analysis (ABA), Early Social Interaction (ESI), floortime, occupational therapy, and pivotal response therapy.

Additionally, there are medical and physical conditions associated with ASD including but not limited to gastrointestinal (GI) issues, epilepsy, feeding issues, and sleep issues that can also be addressed more efficiently if the newborn has been identified or diagnosed with ASD.

### EXAMPLES

The present invention may be better understood by reference to the following non-limiting examples, which are presented in order to more fully illustrate the preferred embodiments of the invention. They should in no way be construed to limit the broad scope of the invention.

### Example 1-Materials and Methods

### Study Subjects

The ABC (11), comprising 804 ASD cases and 1,786 randomly selected pool of control subjects is nested within the Norwegian Mother, Father, and Child Cohort (MoBa) (12, 13), a population-based pregnancy cohort comprising 114,473 children born in 1999-2009, with 95,244 mothers and more than 75,000 fathers (12).

### ASD Case Ascertainment

Children with ASD were identified through questionnaire screening of mothers at offspring of 3, 5, and 7 years of age; referrals of participants suspected of ASD; and annual linkage to the Norwegian Patient Register (NPR) (14). Cases were defined according to the Diagnostic and Statistical Manual of Mental Disorders, Fourth Edition, Text Revision (DSM-IV-TR) (15) criteria. Children diagnosed at the ABC Study Clinic were assessed by clinical psychologists and child psychiatrists using standardized diagnostic instruments including the Autism Diagnostic Interview-Revised (ADI-R) (16) and the Autism Diagnostic Observation Schedule (ADOS) (17) and tests of intellectual and adaptive functioning and language capacity. ASD cases among NPR-identified children not evaluated at the ABC Study Clinic were those assigned International Classification of Diseases, Tenth Revision (ICD-10) F84 diagnoses (18). NPR ASD diagnoses have high validity with a positive predictive value of 96.7% (14).

### Study Sample Selection

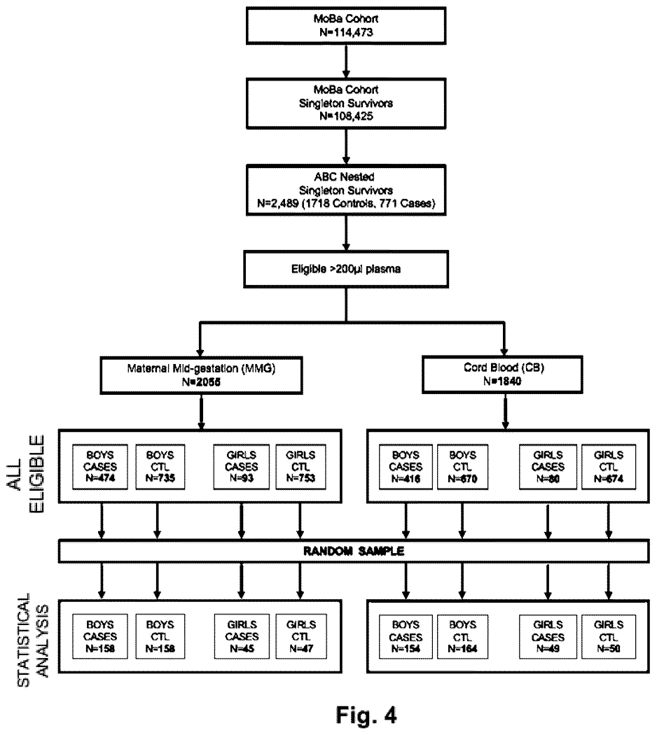

Inclusion criteria included singleton birth, continued participation in the cohort, survival to at least age three years, and availability of 200 microliters or more of MMG and/or umbilical CB plasma. We prioritized subjects with both MMG and CB samples for the metabolomics analyses. For each sex- and sample type-stratified group, we included all available MMG and CB samples from ASD cases that satisfied the inclusion criteria, and the control samples were randomly selected from the pool of eligible controls (FIG. 4).

### Covariates

Data on covariates were extracted from MoBa questionnaires. For MMG analyses, data from the questionnaires covered the time period during pregnancy up to the MMG blood draw; for CB analyses, data covered the entire pregnancy. Confounders included maternal age; maternal report of fever; respiratory or other infection; autoimmune/allergic disorders; emotional distress ratings (Hopkins Symptom Check List) (SCL-5) (19); and use of antipyretics (ATC codes N02BE01, N02BA01, N02BA51, N02BB51). For MMG analyses, we adjusted for gestational age at the MMG blood draw. For both MMG and CB analyses, we conducted sensitivity analyses based on covariates of parental education, maternal exposure to folic acid supplements between 0-8 weeks of gestation, and maternal reports of intake of selective serotonin reuptake inhibitors (SSRIs).

### MMG and CB Plasma Collection

MMG plasma samples were collected during the routine ultrasound examinations at 18 weeks of gestation. Umbilical CB plasma samples were collected at birth. Samples were stored at −80° C. (13, 20).

### Metabolomics Assays

For MMG and CB analyses, samples were sequentially run in an order that alternated based on ASD outcome and sex. Untargeted metabolomics data were acquired using three chromatography/mass spectrometry-based assays (MS): (1) Primary metabolites such as mono- and disaccharides, hydroxyl- and amino acids were measured by gas chromatography/time-of-flight mass spectrometry (GC-TOF MS) (21) including data alignment and compound annotation using the BinBase database algorithm (22); (2) Biogenic amines including microbial compounds such as trimethylamine N-oxide (TMAO), methylated and acetylated amino acids, and short di- and tripeptides were measured by hydrophilic interaction liquid chromatography/quadrupole time-of-flight mass spectrometry (HILIC-QTOF MS); (3) Complex lipids including phosphoglycerolipids, triacylglycerides, sphingolipids, and free fatty acids were analyzed by liquid chromatography (LC)/quadrupole time-of-flight mass spectrometry (CSH-QTOF MS) (23). Targeted bioactive oxylipin assay included thromboxanes, prostaglandins, and hydroxy-, keto- and epoxy-lipins. All LC-MS/MS data included diverse sets of internal standards. LC-MS data were processed by MS-DIAL vs. 4.0 software (24), and compounds annotated based on accurate mass, retention time and MS/MS fragment matching using LipidBlast (25) and Massbank of North America libraries (26). MS-FLO was used to remove erroneous peaks and reduce the false discovery rate in LC datasets (27). A total of 1 208 metabolites were annotated, including 146 primary metabolites (PM), 416 biogenic amines (BA), 577 complex lipids (CL), and 69 oxylipins (OL). Data were normalized by SERRF (28). Residual technical errors were assessed by coefficients of variation for known metabolites.

### Statistical Analyses

For each analyte, missing values reflecting measurements below the detection limit were replaced with 50% of the smallest available value. For each of the sex- and sample type-stratified study cohorts, we identified outliers in each of the four panels using principal component analysis (PCA) (Table 4). After eliminating outliers, data were natural log-transformed and divided by the standard deviation of the analyte within the control group.

Adjusted logistic regression models were used to test separately in boys and girls for associations between metabolites and ASD. In both MMG and CB analyses, models were adjusted for maternal age, illnesses (fever, infection, inflammatory, autoimmune, allergic disorders), emotional distress scores (SCL-5), and non-NSAID antipyretic medications, as well as gestational age. Multiple comparisons were corrected using the Benjamini-Hochberg procedure, controlling the overall false discovery rate (FDR) at the level of 0.05. Additionally, chemical enrichment analyses (ChemRICH) (29) were performed to determine chemical classes that were altered between groups. ChemRICH does not rely upon background databases for statistical calculations and provides enrichment analysis based upon chemical structure, as opposed to defined pathways that can be inherently flawed (29). For each metabolite, we conducted Bayesian analysis on the logistic regression models. The Bayesian alternatives to the null hypothesis significance testing (NHST) framework has been proven to improve the biological interpretability of metabolomics data from human cohorts (30). We then calculated the BF and 95% highest density credible intervals (HDI). We considered a metabolite to be associated with ASD if BF>10 and the 95% credible interval did not overlap with zero (31) (see below Bayesian Analyses).

To examine sex differences in metabolomic profiles, we combined data from boys and girls and repeated the adjusted logistic regression models including sex as a covariate, together with the interaction term between sex and each metabolite. ChemRICH analyses were conducted using the estimates of the sex-metabolite interaction terms.

To explore the utility of the metabolomics assay as a biomarker for ASD, we employed four machine learning algorithms: least absolute shrinkage and selection operator (Lasso) (32), adaptive Lasso (AdaLasso) (33), Random Forests (RF) (34), and XGBoost (35). Models were built, trained, and evaluated separately for each sample type (MMG, CB), and sex. Due to the high dimensionality in the metabolomics data, we applied a novel feature selection method called model-X (MX) knockoffs (36) that distinguish important variables from noise in high-dimensional datasets while controlling the FDR. The procedure was repeated for 500 iterations controlling the FDR at the level of 0.1, and the variable importance was measured as the number of iterations in which a metabolomic analyte was selected (see below, MX Knockoffs). For each of the machine learning algorithms, we considered four sets of predictors: Set 1, all metabolites; Set 2, metabolites with BF>3; Set 3, metabolites that were selected by MX knockoffs in more than one iteration; Set 4, metabolites that were selected by MX knockoffs in at least one iteration and had BF>1. The predictive performance was evaluated in random sampling cross-validation with 500 iterations. In each iteration, the sample set was randomly divided into an 80% training set and a 20% test set. We generated Receiver Operating Characteristic (ROC) curves and then calculated the Area under the Receiver Operating Characteristic (AUROC) curve.

Data analyses were conducted using Matlab (R2021a, The Mathworks Inc., MA) and R (version 4.1.0). All p-values were 2-tailed.

### Batch Effect Correction

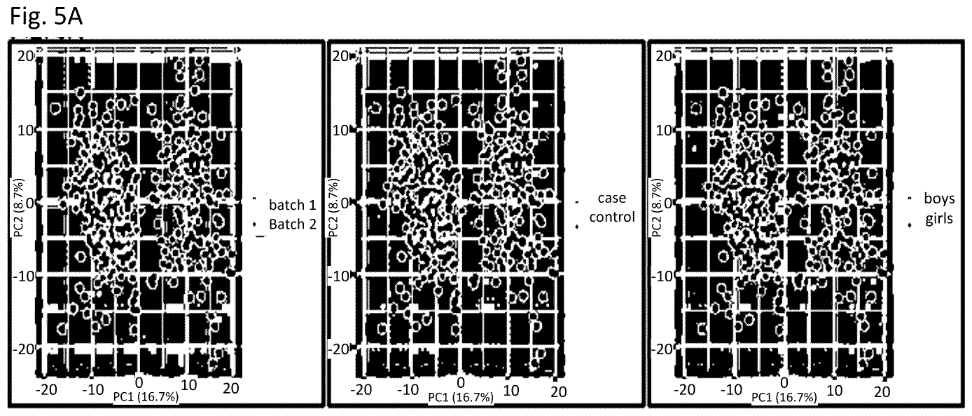

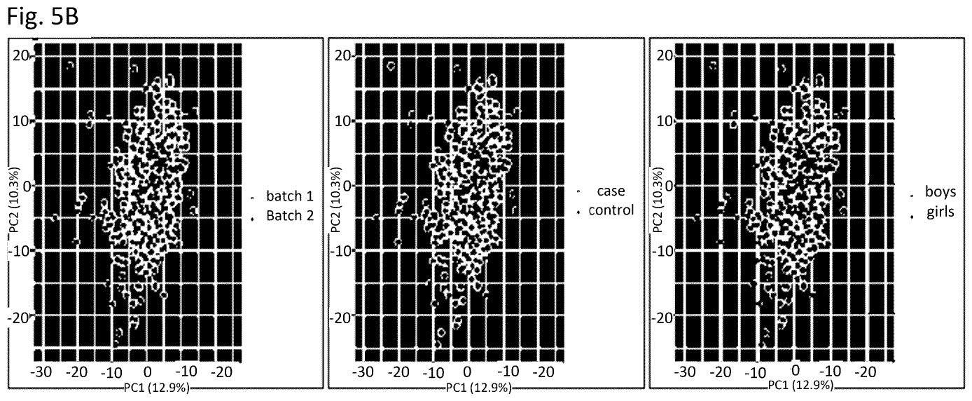

Using scatter plots of the principal components, we detected a batch effect in the complex lipid (CL) data from maternal mid-gestation (MMG) plasma samples that was linked to equipment failure resulting in a one-month delay in completing the lipidomic analyses (FIG. 5A). After encountering batch effects in transcriptomic studies, we adopted a strategy for correction whereby analyses of specimens from cases and controls alternate. In this instance, we assembled quartets comprising (in order) one autism spectrum disorder (ASD) boy, one control boy, one ASD girl, and one control girl. The individual quartets were then randomized and submitted for metabolomic analysis. We identified 3 metabolites for which the prevalence of values below the detection limit differed dramatically between batches: PC (34:0)-ESI (−) (0% in batch 1, 48.8% in batch 2), LPE (20:4)-ESI (+) (4.3% in batch 1, 48.4% in batch 2), and PC (p-40:7)/PC (0-40:8) (49.4% in batch 1, 29.5% in batch 2). The difference in prevalence did not exceed 5% for other metabolites. Accordingly, PC (34:0)-ESI (−), LPE (20:4)-ESI (+), and PC (p-40:7)/PC (0-40:8) were excluded from MMG analyses. Comparisons of variance between batches suggested that mere shifting the means or medians was not sufficient, therefore, we implemented ComBat that uses a Bayesian framework to correct for the batch effect. FIG. 5B shows that the correction was successful.

### Bayesian Analyses

For each metabolite, we conducted Bayesian analysis on the logistic regression models using R packages “rstanarm” and “bayestestR” [111]. The Bayesian alternatives to the null hypothesis significance testing (NHST) framework has been proven to improve the biological interpretability of metabolomics data from human cohorts [112]. Specifically, under the frequentist NHST framework, univariate metabolomic analyses using false discovery rate (FDR) or family-wise error rate (FWER) procedure only consider a metabolite to be associated with the outcome if its p-value falls below an arbitrary threshold. However, NHST cannot distinguish whether there is a true null effect or the data are insensitive. Moreover, a p-value reports the probability of the data given the hypothesis, rather than the probability of the hypothesis given the data. Thus, analysis under the frequentist framework cannot provide support for an alternative hypothesis. On the other hand, univariate metabolomic analyses under Bayesian framework can quantify the size of an effect and the strength of evidence in favor of one hypothesis over another through the estimation of BayesFactor (BF). This allows investigators to discriminate between an inconclusive finding and evidence in favor of the null hypothesis. In our Bayesian analyses, default (weakly informative) prior distributions from rstanarm were applied adjusting the scales of the priors internally. The default priors do not strongly affect the posterior distribution but help stabilize computation, while still allowing for extreme effect sizes if warranted by the data [113, 114]. We then calculated the BFs and 95% highest density credible intervals (HDIs). BFs are ratios that quantify the probability of the alternative hypothesis (B/0) over the null hypothesis (B=0) by estimating the strength of evidence [115]. Therefore, BF=1 indicates equal likelihoods of either hypothesis, given the data. Values larger than 1 imply stronger evidence in favor of the alternative hypothesis over the null hypothesis. We used Jeffreys' guidelines to categorize these values as anecdotal (BF between 1 and 3), moderate (BF between 3 and 10), strong (BF between 10 and 30), very strong (BF between 30 and 100), or extreme (BF>100) evidence for the alternative hypothesis. We considered a metabolite to be associated with ASD if BF>10. The 95% credible intervals are a range of values within which the true effect falls at 95% confidence, given the data [117]. It has a different interpretation from the 95% confidence interval (CI) in the frequentist framework which instead signifies that with a large number of repeated samples, 95% of such calculated CIs would include the true value of the parameter. Metabolites were considered to be altered if the 95% credible interval did not overlap with zero.

### MX Knockoffs

To explore the utility of the metabolomics assay as a biomarker for ASD, we applied a novel feature selection method called “model-X” knockoffs or MX knockoffs due to the high dimensionality in the metabolomics data. MX knockoffs teases apart the important variables from noise in high-dimensional datasets while controlling the FDR. The basic idea behind the approach is to generate a set of “control” variables—the eponymous knockoffs-against which to test the importance of the original input features. The method has mostly been employed in genetic association studies [119-120], but has recently drawn attention in metabolomics [121, 122]. The MX knockoff procedure can be conducted in the parametric setting using Lasso and in the nonparametric setting using Random Forests (RF). We used MX knockoffs with RF because the covariate distribution of the metabolomics data is unknown. The procedure was repeated for 500 iterations controlling the FDR at the level of 0.1, and the variable importance was measured as the number of iterations in which a metabolomic analyte was selected.

### Example 2-Study Population Characteristics

MMG analyses included 408 samples from 158 ASD boys, 158 control boys, 45 ASD girls, and 47 control girls. CB analyses included 418 samples from 155 ASD boys, 164 control boys, 49 ASD girls, and 50 control girls. Demographic and clinical characteristics are illustrated in Table 1. Birth year was differently proportioned between ASD and control boys in the CB analysis (chi-squaredp=0.010). The distribution of birth season was different between ASD and control boys in both MMG (chi-squaredp=0.049) and CB (chi-squaredp=0.020) analyses. In CB analysis, ASD boys were more likely to be born outside of the 37-41 gestational week window (chi-squaredp=0.015). Other parameters did not significantly differ between cases and controls.

### Example 3-Metabolomic Datasets

Targeted and untargeted mass spectrometry platforms yielded data for 1,208 metabolic analytes comprising 146 PM, 416 BA, 577 CL, and 69 OL. We detected a batch effect in the CL data from MMG samples (FIG. 5A). Through quality control, we eliminated three metabolites (PC (34:0)-ESI (−), LPE (20:4)-ESI (+), and PC (p-40:7)/PC (0-40:8)) in the MMG analysis due to dramatic difference in the missing value prevalence between batches. Using ComBat (37), we corrected for the batch effect (FIG. 5B).

### Example 4-ASD is Associated with Altered Metabolomic Profiles

Table 2 shows the estimated adjusted odds ratios (aOR), associated 95% confidence intervals (CI), p-values, FDR adjusted p-values, and BFs (31) of the metabolites associated with ASD risk in each of the sex- and sample type-stratified datasets. Because we used weakly informative priors in Bayesian analysis, the maximum-a-posteriori estimates of the aORs (aOR_{MAP}) and the 95% HDIs were similar to the aORs and 95% CIs, respectively.

In MMG, no metabolite was associated with ASD risk under the NHST framework (all FDR adjusted p-values were above 0.05). Using Bayesian analysis, we found ASD associations (BF>10) in girls with decreased levels of 2,6-dihydroxybenzoic acid and increased levels of glutamic acid, pyroglutamic acid, propionic acid, N-methylalanine, and pseudouridine in the BA panel. In the CL panel, ASD girls had higher levels of PC (p-40:3)/PC (0-40:4) and HexCer-NS (d34: 1) than control girls; and the levels of TAG (56:9), PE (36:5)/PE (16:0-20:5), PC (40:8)-ESI (+), and PC (37:5) were lower in ASD girls compared to control girls.

In MMG boys, Bayesian analysis identified increased levels of FA (20:3) (homo-gamma-linolenic acid) and OxPC (34: 2+1O)/OxPC (16:0-18: 2+1O) in the CL panel to be associated with increased ASD risk. Levels of 17-hydroxy-4,7,10,13,15,19-docosahexaenoic acid in the OL panel were lower in ASD boys.

In CB, through Bayesian analysis, we found increased levels of orotic acid in the PM panel and 2′-O-methylguanosine in the BA panel in ASD girls. In the CL panel, ASD girls had higher levels of DAG (36:1), TAG (56:3), and FA (20:3) (eicosatrienoic acid). Levels of TAG (56:9), TAG (56:8) A, and TAG (58:10) were reduced. Compared to controls, ASD boys had higher levels of FA (20:4) (arachidonic acid, AA) and erythritol under both NHST and Bayesian frameworks (FDR adjusted p<0.05 and BF>10). Bayesian analysis additionally identified ASD association with increased levels of alanine, glucose-6-phosphate, pseudouridine, methionine, and succinic acid in the PM panel in cord blood samples from the male subjects. ASD boys had higher levels of 2′-deoxyadenosine-5′-monophosphate and glycerophosphocholine, and lower levels of 1,4-cyclohexanedione and glutamine. Levels of Leukotriene B4 in the OL panel were significantly reduced in ASD boys.

We also compared ratios of AA/Docosahexaenoic acid (DHA), AA/Eicosapentaenoic acid (EPA) (38, 39), and glutamate (Glu)/glutamine (Gln) (40, 41) between ASD and control subjects in each of the sex- and sample type-stratified datasets (Table 5). In MMG, AA/EPA ratios were elevated in ASD girls, but not in boys. In CB, both boys and girls with ASD had elevated AA/DHA ratios. Glu/Gln ratios were elevated in both ASD boys and girls in MMG. In CB, ASD boys, but not girls, had elevated Glu/Gln ratios.

### Example 5-Set Enrichment Analysis Revealed Altered Chemical Clusters in ASD

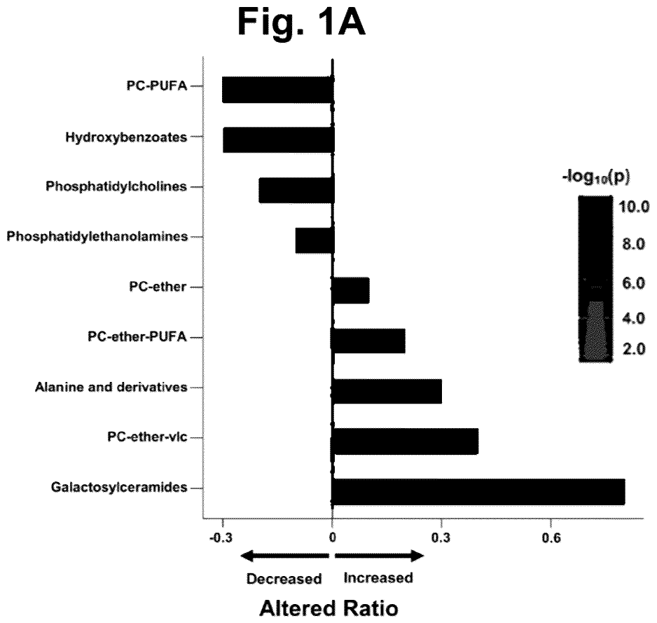

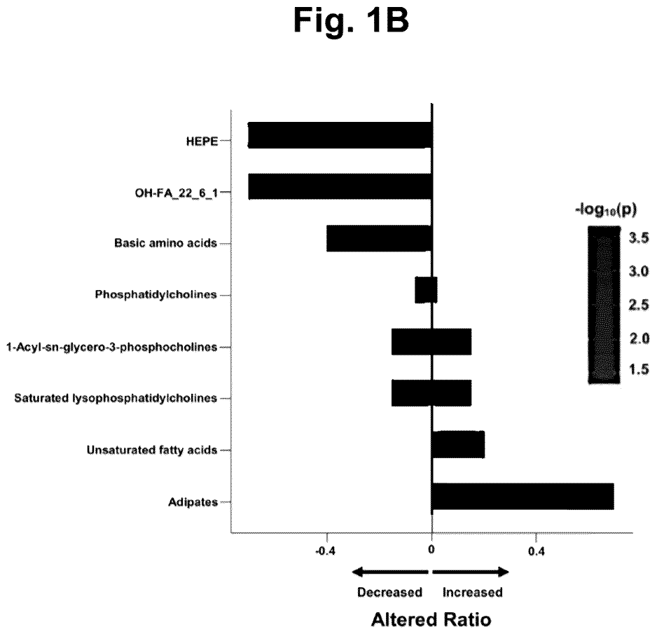

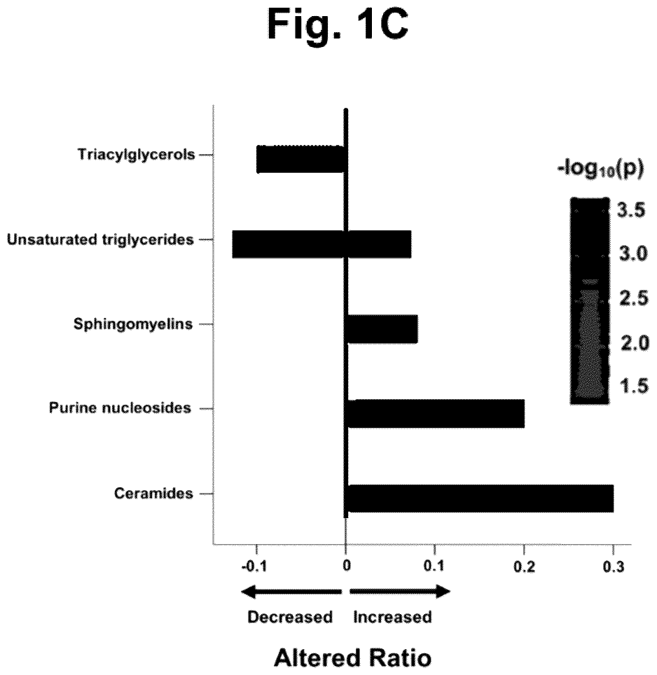

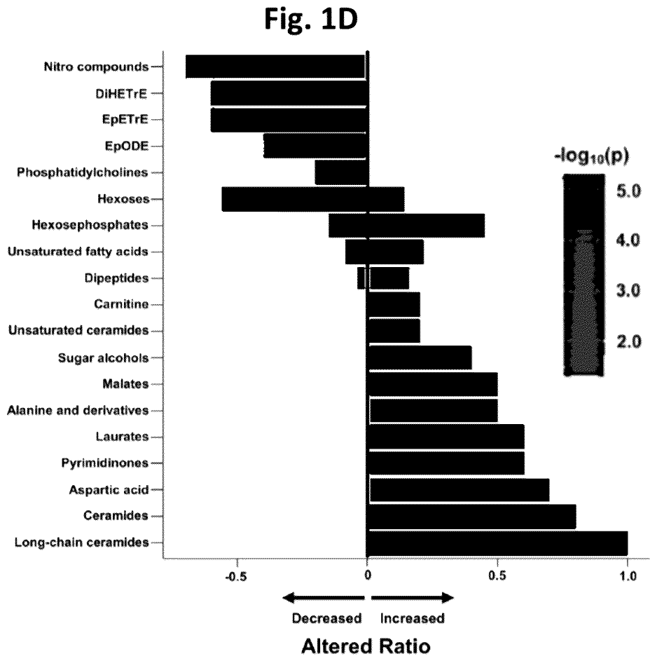

Chemical enrichment analyses of the results from the logistic regression models were performed using ChemRICH (29) to determine chemical clusters that were altered between ASD and control groups (FIG. 1A-1D, Table 3).

In MMG, ASD girls had reduced levels of polyunsaturated fatty acid-containing phosphatidylcholine (PC-PUFA), hydroxybenzoates, phosphatidylcholines (PC), and phosphatidylethanolamines (PE). Increased levels of galactosylceramides, very long-chain ether-linked phosphatidylcholine (PC-ether-vlc), alanine and derivatives, polyunsaturated fatty acid-containing ether-linked phosphatidylcholine (PC-ether-PUFA), and ether-linked phosphatidylcholine (PC-ether) were associated with increased risk of ASD (FIG. 1A). ASD boys had lower levels of hydroxy eicosapentaenoic acid (HEPE), hydroxy fatty acid_22_6_1 (OH-FA_22_6_1), and basic amino acids. There were mixed directional alterations in the levels of PCs, 1-acyl-sn-glycero-3-phosphocholines, and saturated lysophosphatidylcholines (LPC). Levels of adipates and unsaturated fatty acids were increased in ASD boys (FIG. 1B).

In CB, levels of triacylglycerols and the majority of unsaturated triglycerides were reduced in ASD girls. ASD girls had increased levels of ceramides, purine nucleosides, and sphingomyelins (SM) (FIG. 1C). ChemRICH analysis of the CB from the boys revealed the highest number of altered chemical clusters (FIG. 1D). Levels of nitro compounds, dihydroxyeicosatrienoic acid (DiHETrE), epoxyeicosatrienoic acid (EpETrE), epoxyoctadecadienoic acid (EpODE), PCs, and the majority of hexoses were lower in ASD boys. The majority of hexosephosphates, unsaturated fatty acids, and dipeptides were up-regulated. ASD boys also had higher levels of long-chain ceramides, ceramides, aspartic acids, pyrimidinones, laurates, alanine and derivatives, malates, sugar alcohols, unsaturated ceramides, and carnitines.

ASD risk in children is associated with parental education (42) and periconceptional maternal intake of folic acid (43). Accordingly, we conducted sensitivity analyses with ChemRICH additionally adjusting for parental education and maternal exposure to folic acid supplements between 0-8 weeks of gestation. No substantive changes in the dysregulated chemical clusters were observed (data not shown).

### Example 6-ASD Boys and Girls have Different Metabolomic Profiles

We combined metabolomic data from boys and girls and repeated the adjusted logistic regression models including sex as a covariate, together with the interaction term between sex and each metabolite. ChemRICH analyses were conducted using the estimates of the sex-metabolite interaction terms. Univariate analyses in boys and girls were used to elucidate the directions and magnitudes of sex differences (Table 2). The full ChemRICH results for these interaction analyses are reported in Table 6.

In MMG, the chemical clusters with significant sex differences in ASD association included PC-PUFA, galactosylceramides, PC-ether-vlc, PC-ether, phosphatidylinositols (PI), PEs, and glycosphingolipids. Compound estimates in boys and girls revealed that the sex-specific differences were driven by the female cohort; there were no differences in levels of the metabolites between ASD and control boys. In contrast, girls with ASD had lower levels of PC-PUFA, PIs, and PEs, and higher levels of galactosylceramides, PC-ether-vlc, PC-ether, and glycosphingolipids.

In CB, we found two chemical clusters with sex differences in ASD association: hexosephosphates and cyclohexanes. The ASD associations with these metabolites were in opposite directions between boys and girls (increased in ASD boys but decreased in ASD girls; or decreased in ASD boys but increased in ASD girls); estimates in the male cohort were more significant.

### Example 7—Assessment of the Metabolomic Assays as a Biomarker for ASD

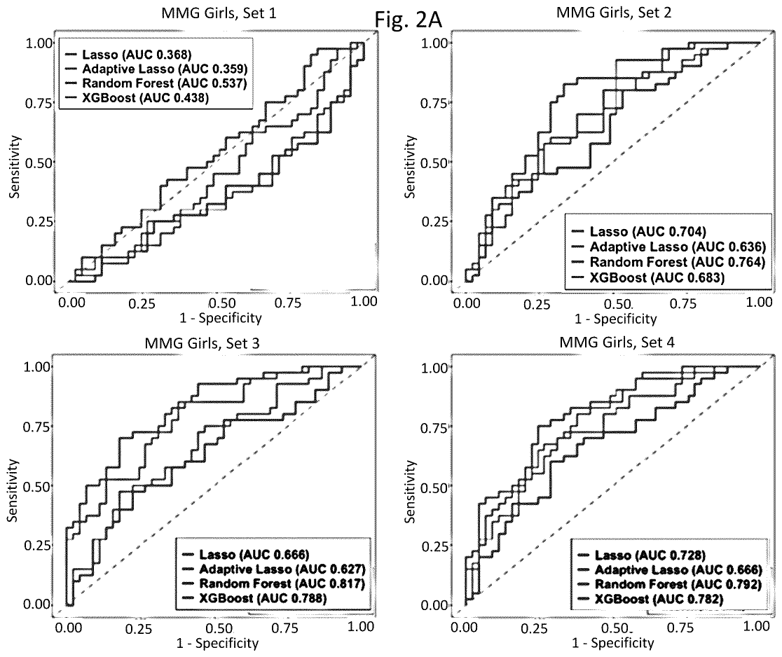

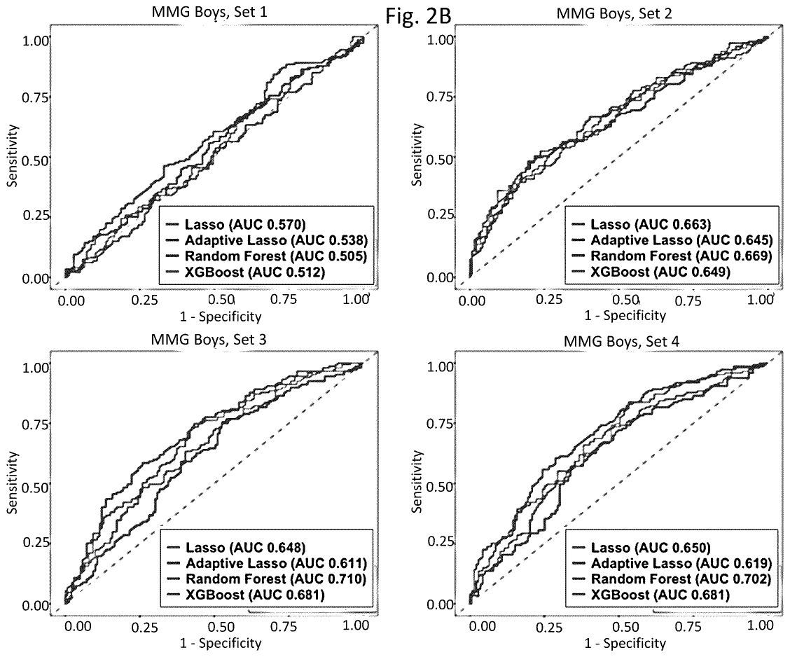

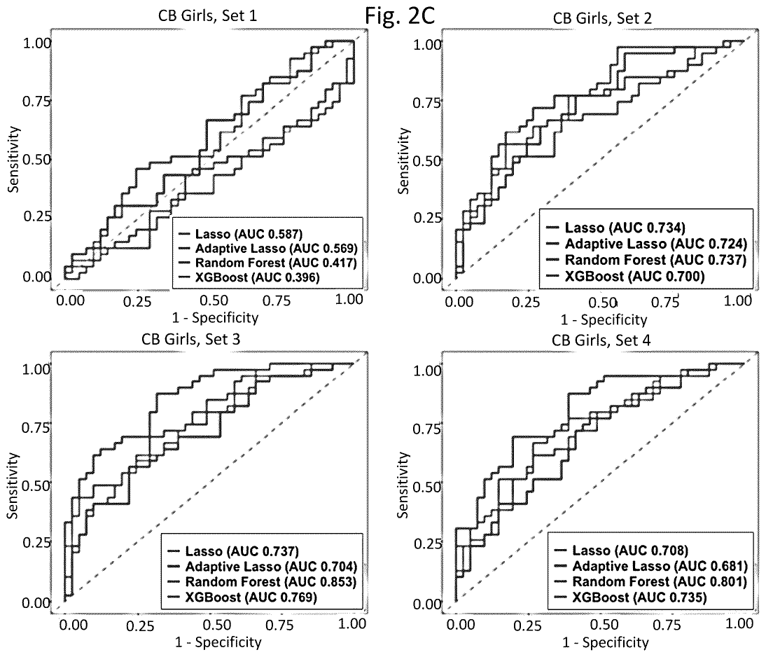

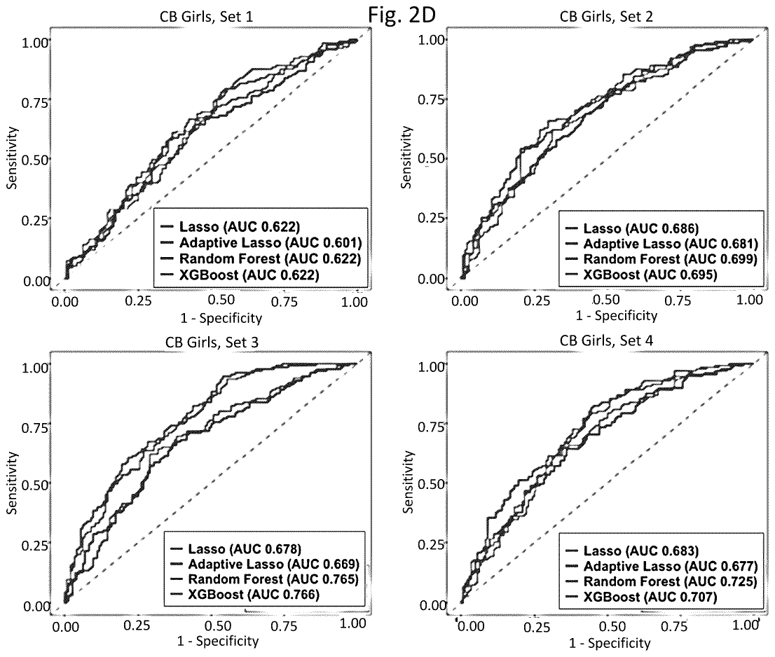

We considered four sets of predictors for each of the machine learning algorithms: Set 1, all metabolites; Set 2, metabolites with BF>3; Set 3, metabolites selected by MX knockoffs in more than one iteration (Table 7); and Set 4, metabolites selected by MX knockoffs in at least one iteration with BF>1. FIG. 2A-D show the ROC curves and the AUROC values, differentiating ASD cases from controls. The AUROC values and 95% CIs are reported in Table 8. The p-values comparing the predictive performances between machine learning algorithms within each predictor set and between predictor sets using each algorithm was also performed (results not shown).

In the MMG girls (FIG. 2A), machine learning models with Set 4 predictors yielded highest average AUROC values (Set 1:0.426; Set 2:0.697; Set 3:0.724; Set 4:0.742); the best performing classifier was RF with Set 3 predictors that differentiated ASD girls from control girls with an AUROC value of 0.817 (95% CI: 0.711 ~ 0.890). In MMG boys (FIG. 2B), machine learning models with Set 2, Set 3, and Set 4 predictors produced similar average AUROC values (Set 1:0.531; Set 2:0.657; Set 3:0.663; Set 4:0.663). RF with Set 3 predictors outperformed the other classifiers and yielded an AUROC value of 0.710 (95% CI: 0.647 ~ 0.766). In CB girls (FIG. 2C), machine learning models with Set 3 predictors were more accurate than models based on the other predictors (Set 1:0.492; Set 2:0.724; Set 3:0.766; Set 4:0.731). RF with Set 3 predictors separated ASD girls from control girls with an AUROC value of 0.853 (95% CI: 0.754 ~ 0.917). In CB boys (FIG. 2D), Set 3 was the most accurate predictors (Set 1:0.617; Set 2:0.690; Set 3:0.720; Set 4:0.698); XGBoost with Set 3 predictors produced an AUROC value of 0.766 (95% CI: 0.707 ~0.816).

### Example 8—Post Hoc Power Analysis

Autism is approximately 4-fold less common in girls. Accordingly, our sample size was smaller for girls than boys. We conducted post hoc power analysis for every logistic regression model and compared power between boys and girls in the MMG and cord blood (CB) datasets, respectively. In the MMG analysis, the female cohort, despite smaller sample size, exhibited higher power than the male cohort (power in girls had a mean of 0.188 and a standard deviation of 0.176 vs. power in boys had a mean of 0.168 and a standard deviation of 0.166, paired t-test p-0.003), suggesting that the effect sizes in girls were larger than those in boys. In the CB analysis, the male cohort had higher power than the female cohort (power in girls had a mean of 0.173 and a standard deviation of 0.162 vs. power in boys had a mean of 0.219 and a standard deviation of 0.219, paired t-test p=5.11×10⁻⁹) (results of post hoc powers for individual metabolites not shown).

### Example 9—Subsampling Analysis

To examine whether the difference in the altered chemical clusters between boys and girls resulted from the difference in the sample sizes, we conducted 1 000 iterations of random subsampling (without replacement) of 50 ASD boys and 50 control boys in the MMG and CB datasets, respectively. The logistic regression estimates from 1,000 subsamples were pooled

using Rubin's rules and used as inputs for ChemRICH. No chemical clusters, in either MMG or CB analyses, were significantly altered (data not shown).

### REFERENCES

1. Maenner M J, Shaw K A, Bakian A V, Bilder D A, Durkin M S, Esler A, et al. Prevalence and Characteristics of Autism Spectrum Disorder Among Children Aged 8 Years-Autism and Developmental Disabilities Monitoring Network, 11 Sites, United States, 2018. MMWR Surveill Summ. 2021; 70 (11): 1-16.
2. Towle P O, Patrick P A, Ridgard T, Pham S, Marrus J. Is Earlier Better? The Relationship between Age When Starting Early Intervention and Outcomes for Children with Autism Spectrum Disorder: A Selective Review. Autism Res Treat. 2020; 2020:7605876.
3. Wallace K S, Rogers S J. Intervening in infancy: implications for autism spectrum disorders. J Child Psychol Psychiatry. 2010; 51 (12): 1300-20.
4. Zwaigenbaum L, Penner M. Autism spectrum disorder: advances in diagnosis and evaluation. BMJ. 2018; 361: k1674.
5. Siu M T, Weksberg R. Epigenetics of Autism Spectrum Disorder. Adv Exp Med Biol. 2017; 978:63-90.
6. Ritz B, Yan Q, Uppal K, Liew Z, Cui X, Ling C, et al. Untargeted Metabolomics Screen of Mid-pregnancy Maternal Serum and Autism in Offspring. Autism Res. 2020; 13 (8): 1258-69.
7. Schmidt R J, Liang D, Busgang S A, Curtin P, Giulivi C. Maternal Plasma Metabolic Profile Demarcates a Role for Neuroinflammation in Non-Typical Development of Children. Metabolites. 2021; 11 (8).
8. Panjwani A A, Ji Y, Fahey J W, Palmer A, Wang G, Hong X, et al. Maternal Dyslipidemia, Plasma Branched-Chain Amino Acids, and the Risk of Child Autism Spectrum Disorder: Evidence of Sex Difference. J Autism Dev Disord. 2020; 50 (2): 540-50.
9. Courraud J, Ernst M, Svane Laursen S, Hougaard D M, Cohen A S. Studying Autism Using Untargeted Metabolomics in Newborn Screening Samples. J Mol Neurosci. 2021; 71 (7): 1378-93.
10. Barone R, Alaimo S, Messina M, Pulvirenti A, Bastin J, Group MI-A, et al. A Subset of Patients With Autism Spectrum Disorders Show a Distinctive Metabolic Profile by Dried Blood Spot Analyses. Front Psychiatry. 2018; 9:636.
11. Stoltenberg C, Schjolberg S, Bresnahan M, Hornig M, Hirtz D, Dahl C, et al. The Autism Birth Cohort: a paradigm for gene-environment-timing research. Mol Psychiatry. 2010; 15 (7): 676-80.
12. Magnus P, Irgens L M, Haug K, Nystad W, Skjerven R, Stoltenberg C, et al. Cohort profile: the Norwegian Mother and Child Cohort Study (MoBa). Int J Epidemiol. 2006; 35 (5): 1146-50.
13. Magnus P, Birke C, Vejrup K, Haugan A, Alsaker E, Daltveit A K, et al. Cohort Profile Update: The Norwegian Mother and Child Cohort Study (MoBa). Int J Epidemiol. 2016; 45 (2): 382-8.
14. Bresnahan M, Hornig M, Schultz A F, Gunnes N, Hirtz D, Lie K K, et al. Association of maternal report of infant and toddler gastrointestinal symptoms with autism: evidence from a prospective birth cohort. JAMA Psychiatry. 2015; 72 (5): 466-74.
15. Association AP. Diagnostic and Statistical Manual of Mental Disorders, Fourth Edition, Text Revision (DSM-IV-TR) . . . . American Psychiatric Association: Arlington. 2000.
16. Lord C, Rutter M, Le Couteur A. Autism Diagnostic Interview-Revised: a revised version of a diagnostic interview for caregivers of individuals with possible pervasive developmental disorders. J Autism Dev Disord. 1994;24 (5): 659-85.
17. Lord C, Risi S, Lambrecht L, Cook E H, Jr., Leventhal B L, DiLavore P C, et al. The autism diagnostic observation schedule-generic: a standard measure of social and communication deficits associated with the spectrum of autism. J Autism Dev Disord. 2000; 30 (3): 205-23.
18. Organization WH. International classification of diseases and related health problems, 10th revision. Geneva: World Health Organization. 1992.
19. Strand B H, Dalgard O S, Tambs K, Rognerud M. Measuring the mental health status of the Norwegian population: a comparison of the instruments SCL-25, SCL-10, SCL-5 and MHI-5 (SF-36). Nord J Psychiatry. 2003; 57 (2): 113-8.
20. Ronningen K S, Paltiel L, Meltzer H M, Nordhagen R, Lie K K, Hovengen R, et al. The biobank of the Norwegian Mother and Child Cohort Study: a resource for the next 100 years. Eur J Epidemiol. 2006; 21 (8): 619-25.
21. Fiehn O. Metabolomics by Gas Chromatography-Mass Spectrometry: Combined Targeted and Untargeted Profiling. Curr Protoc Mol Biol. 2016; 114:30 4 1-4 2.
22. Kind T, Wohlgemuth G, Lee D Y, Lu Y, Palazoglu M, Shahbaz S, et al. FiehnLib: mass spectral and retention index libraries for metabolomics based on quadrupole and time-of-flight gas chromatography/mass spectrometry. Anal Chem. 2009; 81 (24): 10038-48.
23. Cajka T, Smilowitz J T, Fiehn O. Validating Quantitative Untargeted Lipidomics Across Nine Liquid Chromatography-High-Resolution Mass Spectrometry Platforms. Anal Chem. 2017; 89 (22): 12360-8.
24. Tsugawa H, Cajka T, Kind T, Ma Y, Higgins B, Ikeda K, et al. MS-DIAL: data-independent MS/MS deconvolution for comprehensive metabolome analysis. Nat Methods. 2015; 12 (6): 523-6.
25. Tsugawa H, Ikeda K, Takahashi M, Satoh A, Mori Y, Uchino H, et al. A lipidome atlas in MS-DIAL 4. Nat Biotechnol. 2020; 38 (10): 1159-63.
26. Wohlgemuth G, Mehta S S, Mejia RF, Neumann S, Pedrosa D, Pluskal T, et al. SPLASH, a hashed identifier for mass spectra. Nat Biotechnol. 2016; 34 (11): 1099-101.
27. DeFelice B C, Mehta S S, Samra S, Cajka T, Wancewicz B, Fahrmann J F, et al. Mass Spectral Feature List Optimizer (MS-FLO): A Tool To Minimize False Positive Peak Reports in Untargeted Liquid Chromatography-Mass Spectroscopy (LC-MS) Data Processing. Anal Chem. 2017; 89 (6): 3250-5.
28. Fan S, Kind T, Cajka T, Hazen S L, Tang W H W, Kaddurah-Daouk R, et al. Systematic Error Removal Using Random Forest for Normalizing Large-Scale Untargeted Lipidomics Data. Anal Chem. 2019; 91 (5): 3590-6.
29. Barupal D K, Fiehn O. Chemical Similarity Enrichment Analysis (ChemRICH) as alternative to biochemical pathway mapping for metabolomic datasets. Sci Rep. 2017; 7 (1): 14567.
30. Brydges C, Che X, Lipkin W I, Fiehn O. Bayesian statistics improves biological interpretability of metabolomics data from human cohorts. bioRxiv. 2022.
31. Jeffreys H. Theory of Probability. 3rd ed. Oxford: Clarendon Press; 1961.
32. Tibshirani R. Regression Shrinkage and Selection Via the Lasso. Journal of the Royal Statistical Society: Series B (Methodological). 1996; 58 (1): 267-88.
33. Zou H. The Adaptive Lasso and Its Oracle Properties. Journal of the American Statistical Association. 2012; 101 (476): 1418-29.
34. Breiman L. Random Forests. Machine Learning. 2001; 45:5-32.
35. Chen T, Guestrin C. XGBoost: A Scalable Tree Boosting System. 22nd ACM SIGKDD International Conference on Knowledge Discovery and Data Mining; Aug. 13-17, 2016; San Francisco, CA2016. p. 785-94.
36. Candes E, Fan Y, Janson L, Lv J. Panning for gold: ‘model-X’ knockoffs for high dimensional controlled variable selection. Journal of the Royal Statistical Society: Series B (Statistical Methodology). 2018; 80 (3): 551-77.
37. Johnson W E, Li C, Rabinovic A. Adjusting batch effects in microarray expression data using empirical Bayes methods. Biostatistics. 2007; 8 (1): 118-27.
38. Cappelletti M, Della Bella S, Ferrazzi E, Mavilio D, Divanovic S. Inflammation and preterm birth. J Leukoc Biol. 2016; 99 (1): 67-78.
39. Parletta N, Niyonsenga T, Duff J. Omega-3 and Omega-6 Polyunsaturated Fatty Acid Levels and Correlations with Symptoms in Children with Attention Deficit Hyperactivity Disorder, Autistic Spectrum Disorder and Typically Developing Controls. PLOS One. 2016; 11 (5): e0156432.
40. Al-Otaish H, Al-Ayadhi L, Bjorklund G, Chirumbolo S, Urbina MA, El-Ansary A. Relationship between absolute and relative ratios of glutamate, glutamine and GABA and severity of autism spectrum disorder. Metab Brain Dis. 2018; 33 (3): 843-54.
41. El-Ansary A. Data of multiple regressions analysis between selected biomarkers related to glutamate excitotoxicity and oxidative stress in Saudi autistic patients. Data Brief. 2016; 7:111-6.
42. Van Meter K C, Christiansen L E, Delwiche L D, Azari R, Carpenter T E, Hertz-Picciotto I. Geographic distribution of autism in California: a retrospective birth cohort analysis. Autism Res. 2010; 3 (1): 19-29.
43. Suren P, Roth C, Bresnahan M, Haugen M, Hornig M, Hirtz D, et al. Association between maternal use of folic acid supplements and risk of autism spectrum disorders in children. JAMA. 2013; 309 (6): 570-7.
44. Wiggs K K, Rickert M E, Sujan A C, Quinn P D, Larsson H, Lichtenstein P, et al. Antiseizure medication use during pregnancy and risk of ASD and ADHD in children. Neurology. 2020; 95 (24): e3232-e40.
45. Stromland K, Nordin V, Miller M, Akerstrom B, Gillberg C. Autism in thalidomide embryopathy: a population study. Dev Med Child Neurol. 1994;36 (4): 351-6.
46. Smith S E, Li J, Garbett K, Mirnics K, Patterson P H. Maternal immune activation alters fetal brain development through interleukin-6. J Neurosci. 2007; 27 (40): 10695-702.
47. Nicolini C, Fahnestock M. The valproic acid-induced rodent model of autism. Exp Neurol. 2018; 299 (Pt A): 217-27.
48. Patterson P H. Maternal infection and immune involvement in autism. Trends Mol Med. 2011; 17 (7): 389-94.
49. Parenti M, Schmidt R J, Ozonoff S, Shin H M, Tancredi D J, Krakowiak P, et al. Maternal Serum and Placental Metabolomes in Association with Prenatal Phthalate Exposure and Neurodevelopmental Outcomes in the MARBLES Cohort. Metabolites. 2022; 12 (9).
50. Hollowood K, Melnyk S, Pavliv O, Evans T, Sides A, Schmidt R J, et al. Maternal metabolic profile predicts high or low risk of an autism pregnancy outcome. Res Autism Spectr Disord. 2018; 56:72-82.
51. Girchenko P, Lahti-Pulkkinen M, Lipsanen J, Heinonen K, Lahti J, Rantalainen V, et al. Maternal early-pregnancy body mass index-associated metabolomic component and mental and behavioral disorders in children. Mol Psychiatry. 2022; 27 (11): 4653-61.
52. Lyall K, Windham G C, Snyder N W, Kuskovsky R, Xu P, Bostwick A, et al. Association Between Midpregnancy Polyunsaturated Fatty Acid Levels and Offspring Autism Spectrum Disorder in a California Population-Based Case-Control Study. Am J Epidemiol. 2021; 190 (2): 265-76.
53. Kim J H, Yan Q, Uppal K, Cui X, Ling C, Walker D I, et al. Metabolomics analysis of maternal serum exposed to high air pollution during pregnancy and risk of autism spectrum disorder in offspring. Environ Res. 2021; 196:110823.
54. Ferdinandusse S, McWalter K, Te Brinke H, L I J, Mooijer P M, Ruiter J P N, et al. An autosomal dominant neurological disorder caused by de novo variants in FAR1 resulting in uncontrolled synthesis of ether lipids. Genet Med. 2021; 23 (4): 740-50.
55. Staps P, Rizzo W B, Vaz FM, Bugiani M, Giera M, Heijs B, et al. Disturbed brain ether lipid metabolism and histology in Sjogren-Larsson syndrome. J Inherit Metab Dis. 2020; 43 (6): 1265-78.
56. Vaz F M, McDermott J H, Alders M, Wortmann S B, Kolker S, Pras-Raves M L, et al. Mutations in PCYT2 disrupt etherlipid biosynthesis and cause a complex hereditary spastic paraplegia. Brain. 2019; 142 (11): 3382-97.
57. Teruya T, Chen Y J, Kondoh H, Fukuji Y, Yanagida M. Whole-blood metabolomics of dementia patients reveal classes of disease-linked metabolites. Proc Natl Acad Sci USA. 2021; 118 (37).
58. Bjornevik K, Zhang Z, O'Reilly E J, Berry J D, Clish C B, Deik A, et al. Prediagnostic plasma metabolomics and the risk of amyotrophic lateral sclerosis. Neurology. 2019; 92 (18): e2089-e100.
59. Yang F, Wu S C, Ling Z X, Chao S, Zhang L J, Yan X M, et al. Altered Plasma Metabolic Profiles in Chinese Patients With Multiple Sclerosis. Front Immunol. 2021; 12:792711.
60. Liao X, Yang J, Wang H, Li Y. Microglia mediated neuroinflammation in autism spectrum disorder. Journal of psychiatric research. 2020; 130:167-76.
61. Gupta S, Ellis S E, Ashar F N, Moes A, Bader J S, Zhan J, et al. Transcriptome analysis reveals dysregulation of innate immune response genes and neuronal activity-dependent genes in autism. Nat Commun. 2014; 5:5748.
62. Balic A, Vlasic D, Zuzul K, Marinovic B, Bukvic Mokos Z. Omega-3 Versus Omega-6 Polyunsaturated Fatty Acids in the Prevention and Treatment of Inflammatory Skin Diseases. Int J Mol Sci. 2020; 21 (3).
63. So J, Wu D, Lichtenstein A H, Tai A K, Matthan N R, Maddipati K R, et al. EPA and DHA differentially modulate monocyte inflammatory response in subjects with chronic inflammation in part via plasma specialized pro-resolving lipid mediators: A randomized, double-blind, crossover study. Atherosclerosis. 2021; 316:90-8.
64. Onodera T, Fukuhara A, Shin J, Hayakawa T, Otsuki M, Shimomura I. Eicosapentaenoic acid and 5-HEPE enhance macrophage-mediated Treg induction in mice. Sci Rep. 2017; 7 (1): 4560.
65. Kishikawa A, Kitaura H, Kimura K, Ogawa S, Qi J, Shen W R, et al. Docosahexaenoic Acid Inhibits Inflammation-Induced Osteoclast Formation and Bone Resorption in vivo Through GPR120 by Inhibiting TNF-alpha Production in Macrophages and Directly Inhibiting Osteoclast Formation. Front Endocrinol (Lausanne). 2019; 10:157.
66. Kikut J, Komorniak N, Zietek M, Palma J, Szczuko M. Inflammation with the participation of arachidonic (AA) and linoleic acid (LA) derivatives (HETEs and HODEs) is necessary in the course of a normal reproductive cycle and pregnancy. J Reprod Immunol. 2020; 141:103177.
67. Rizzo M T, Carlo-Stella C. Arachidonic acid mediates interleukin-1 and tumor necrosis factor-alpha-induced activation of the c-jun amino-terminal kinases in stromal cells. Blood. 1996; 88 (10): 3792-800.
68. Hughes-Fulford M, Li C F, Boonyaratanakornkit J, Sayyah S. Arachidonic acid activates phosphatidylinositol 3-kinase signaling and induces gene expression in prostate cancer. Cancer Res. 2006; 66 (3): 1427-33.
69. Node K, Huo Y, Ruan X, Yang B, Spiecker M, Ley K, et al. Anti-inflammatory properties of cytochrome P450 epoxygenase-derived eicosanoids. Science. 1999;285 (5431): 1276-9.
70. Ghazali R, Mehta K J, Bligh S A, Tewfik I, Clemens D, Patel V B. High omega arachidonic acid/docosahexaenoic acid ratio induces mitochondrial dysfunction and altered lipid metabolism in human hepatoma cells. World J Hepatol. 2020; 12 (3): 84-98.
71. Cotogni P, Muzio G, Trombetta A, Ranieri V M, Canuto R A. Impact of the omega-3 to omega-6 polyunsaturated fatty acid ratio on cytokine release in human alveolar cells. JPEN J Parenter Enteral Nutr. 2011; 35 (1): 114-21.
72. Che X, Hornig M, Bresnahan M, Stoltenberg C, Magnus P, Suren P, et al. Maternal mid-gestational and child cord blood immune signatures are strongly associated with offspring risk of ASD. Mol Psychiatry. 2022; 27 (3): 1527-41.
73. Dawaliby R, Trubbia C, Delporte C, Masureel M, Van Antwerpen P, Kobilka B K, et al. Allosteric regulation of G protein-coupled receptor activity by phospholipids. Nat Chem Biol. 2016; 12 (1): 35-9.
74. van der Veen J N, Kennelly J P, Wan S, Vance J E, Vance D E, Jacobs R L. The critical role of phosphatidylcholine and phosphatidylethanolamine metabolism in health and disease. Biochim Biophys Acta Biomembr. 2017; 1859 (9 Pt B): 1558-72.
75. Schuler M H, Di Bartolomeo F, Bottinger L, Horvath S E, Wenz L S, Daum G, et al. Phosphatidylcholine affects the role of the sorting and assembly machinery in the biogenesis of mitochondrial beta-barrel proteins. J Biol Chem. 2015; 290 (44): 26523-32.
76. Niebergall L J, Vance D E. The ratio of phosphatidylcholine to phosphatidylethanolamine does not predict integrity of growing MT58 Chinese hamster ovary cells. Biochim Biophys Acta. 2012; 1821 (2): 324-34.
77. Kano-Sueoka T, Nicks M E. Abnormal function of protein kinase C in cells having phosphatidylethanolamine-deficient and phosphatidylcholine-excess membranes. Cell Growth Differ. 1993;4 (7): 533-7.
78. Rockenfeller P, Koska M, Pietrocola F, Minois N, Knittelfelder O, Sica V, et al. Phosphatidylethanolamine positively regulates autophagy and longevity. Cell Death Differ. 2015; 22 (3): 499-508.
79. Longo N, Frigeni M, Pasquali M. Carnitine transport and fatty acid oxidation. Biochim Biophys Acta. 2016; 1863 (10): 2422-35.
80. Needham B D, Adame M D, Serena G, Rose D R, Preston G M, Conrad M C, et al. Plasma and Fecal Metabolite Profiles in Autism Spectrum Disorder. Biol Psychiatry. 2021; 89 (5): 451-62.
81. Thomas R H, Foley K A, Mepham J R, Tichenoff L J, Possmayer F, MacFabe D F. Altered brain phospholipid and acylcarnitine profiles in propionic acid infused rodents: further development of a potential model of autism spectrum disorders. J Neurochem. 2010; 113 (2): 515-29.
82. Uche L E, Gooris G S, Bouwstra J A, Beddoes C M. Increased Levels of Short-Chain Ceramides Modify the Lipid Organization and Reduce the Lipid Barrier of Skin Model Membranes. Langmuir. 2021; 37 (31): 9478-89.
83. Hussain M M, Jin W, Jiang X C. Mechanisms involved in cellular ceramide homeostasis. Nutr Metab (Lond). 2012; 9 (1): 71.
84. Melland-Smith M, Ermini L, Chauvin S, Craig-Barnes H, Tagliaferro A, Todros T, et al. Disruption of sphingolipid metabolism augments ceramide-induced autophagy in preeclampsia. Autophagy. 2015; 11 (4): 653-69.
85. Ross M M, Piorczynski T B, Harvey J, Burnham T S, Francis M, Larsen M W, et al. Ceramide: a novel inducer for neural tube defects. Dev Dyn. 2019; 248 (10): 979-96.
86. Ariga T, Jarvis W D, Yu R K. Role of sphingolipid-mediated cell death in neurodegenerative diseases. J Lipid Res. 1998;39 (1): 1-16.
87. Stoica B A, Movsesyan V A, Lea PMt, Faden A I. Ceramide-induced neuronal apoptosis is associated with dephosphorylation of Akt, BAD, FKHR, GSK-3beta, and induction of the mitochondrial-dependent intrinsic caspase pathway. Mol Cell Neurosci. 2003; 22 (3): 365-82.
88. France-Lanord V, Brugg B, Michel P P, Agid Y, Ruberg M. Mitochondrial free radical signal in ceramide-dependent apoptosis: a putative mechanism for neuronal death in Parkinson's disease. J Neurochem. 1997;69 (4): 1612-21.
89. Fanani M L, Maggio B. The many faces (and phases) of ceramide and sphingomyelin I single lipids. Biophys Rev. 2017; 9 (5): 589-600.
90. Rogasevskaia T, Coorssen J R. Sphingomyelin-enriched microdomains define the efficiency of native Ca (2+)-triggered membrane fusion. J Cell Sci. 2006; 119 (Pt 13): 2688-94.
91. Corcelle-Termeau E, Vindelov S D, Hamalisto S, Mograbi B, Keldsbo A, Brasen J H, et al. Excess sphingomyelin disturbs ATG9A trafficking and autophagosome closure. Autophagy. 2016; 12 (5): 833-49.
92. MahmoudianDehkordi S, Ahmed A T, Bhattacharyya S, Han X, Baillie R A, Arnold M, et al. Alterations in acylcarnitines, amines, and lipids inform about the mechanism of action of citalopram/escitalopram in major depression. Transl Psychiatry. 2021; 11 (1): 153.
93. Bhattacharyya S, Ahmed A T, Arnold M, Liu D, Luo C, Zhu H, et al. Metabolomic signature of exposure and response to citalopram/escitalopram in depressed outpatients. Transl Psychiatry. 2019; 9 (1): 173.
94. Dorninger F, Forss-Petter S, Berger J. From peroxisomal disorders to common neurodegenerative diseases—the role of ether phospholipids in the nervous system. FEBS Lett. 2017; 591 (18): 2761-88.
95. Frazier T W, Georgiades S, Bishop S L, Hardan A Y. Behavioral and cognitive characteristics of females and males with autism in the Simons Simplex Collection. J Am Acad Child Adolesc Psychiatry. 2014; 53 (3): 329-40 e1-3.
96. Aldred S, Moore K M, Fitzgerald M, Waring R H. Plasma amino acid levels in children with autism and their families. J Autism Dev Disord. 2003; 33 (1): 93-7.
97. Shimmura C, Suda S, Tsuchiya K J, Hashimoto K, Ohno K, Matsuzaki H, et al. Alteration of plasma glutamate and glutamine levels in children with high-functioning autism. PLOS One. 2011; 6 (10): e25340.
98. Belov Kirdajova D, Kriska J, Tureckova J, Anderova M. Ischemia-Triggered Glutamate Excitotoxicity From the Perspective of Glial Cells. Front Cell Neurosci. 2020; 14:51.
99. Tseng E E, Brock M V, Lange M S, Troncoso J C, Blue M E, Lowenstein C J, et al. Glutamate excitotoxicity mediates neuronal apoptosis after hypothermic circulatory arrest. Ann Thorac Surg. 2010; 89 (2): 440-5.
100. Ankarcrona M, Dypbukt J M, Bonfoco E, Zhivotovsky B, Orrenius S, Lipton S A, et al. Glutamate-induced neuronal death: a succession of necrosis or apoptosis depending on mitochondrial function. Neuron. 1995;15 (4): 961-73.
101. Takeuchi H, Jin S, Wang J, Zhang G, Kawanokuchi J, Kuno R, et al. Tumor necrosis factor-alpha induces neurotoxicity via glutamate release from hemichannels of activated microglia in an autocrine manner. J Biol Chem. 2006; 281 (30): 21362-8.
102. Yang G Y, Gong C, Qin Z, Liu X H, Lorris Betz A. Tumor necrosis factor alpha expression produces increased blood-brain barrier permeability following temporary focal cerebral ischemia in mice. Brain Res Mol Brain Res. 1999;69 (1): 135-43.
103. Zhou Y, Danbolt N C. Glutamate as a neurotransmitter in the healthy brain. J Neural Transm (Vienna). 2014; 121 (8): 799-817.
104. Hassan T H, Abdelrahman H M, Fattah N R A, El-Masry N M, Hashim H M, El-Gerby K M, et al. Blood and brain glutamate levels in children with autistic disorder. Research in Autism Spectrum Disorders. 2013; 7 (4): 541-8.
105. Albrecht J, Sidoryk-Wegrzynowicz M, Zielinska M, Aschner M. Roles of glutamine in neurotransmission. Neuron Glia Biol. 2010; 6 (4): 263-76.
106. Chen J, Herrup K. Glutamine acts as a neuroprotectant against DNA damage, beta-amyloid and H202-induced stress. PLOS One. 2012; 7 (3): e33177.
107. Stelmashook E V, Lozier E R, Goryacheva E S, Mergenthaler P, Novikova S V, Zorov D B, et al. Glutamine-mediated protection from neuronal cell death depends on mitochondrial activity. Neurosci Lett. 2010; 482 (2): 151-5.
108. Budni J, Molz S, Dal-Cim T, Martin-de-Saavedra M D, Egea J, Lopez M G, et al. Folic Acid Protects Against Glutamate-Induced Excitotoxicity in Hippocampal Slices Through a Mechanism that Implicates Inhibition of GSK-3beta and iNOS. Mol Neurobiol. 2018; 55 (2): 1580-9.
109. Johnson W E, Li C, Rabinovic A. Adjusting batch effects in microarray expression data using empirical Bayes methods. Biostatistics. 2007; 8 (1): 118-127.
110. Goodrich B, Gabry J, Ali I, Brilleman S. rstanarm: Bayesian applied regression modeling via Stan. R package version. 2020; 2 (1).
111. Makowski D, Ben-Shachar MS, Lüdecke D. bayestestR: Describing effects and their uncertainty, existence and significance within the Bayesian framework. J Open Source Softw. 2019; 4 (40): 1541.
112. Brydges C, Che X, Lipkin W I, Fiehn O. Bayesian statistics improvesbiological interpretability of metabolomics data from human cohorts. bioRxiv. 2022.
113. Gelman A, Jakulin A, Pittau M G, Su Y-S. A weakly informative default prior distribution for logistic and other regression models. Ann Appl Stat. 2008; 2 (4): 1360-1383.
114. Muth C, Oravecz Z, Gabry J. User-friendly Bayesian regression modeling: A tutorial with rstanarm and shinystan. Quant Meth Psych. 2018; 14 (2): 99-119.
115. Lee M D, Wagenmakers E-J. Bayesian cognitive modeling: A practical course. Cambridge university Press; 2014.
116. Jeffreys H. Theory of Probability. 3rd ed. Clarendon Press: Oxford; 1961.
117. Candes E, Fan Y, Janson L, Lv J. Panning for gold: ‘model-X’ knockoffs for high dimensional controlled variable selection. J R Stat Soc Series B Stat Methodol. 2018; 80 (3): 551-577.
118. He T, Baik J M, Kato C, Yang H, Fan Z, Cham J, et al. Novel Ensemble Feature Selection Approach and Application in Repertoire Sequencing Data. Front Genet. 2022; 13:821832.
119. Chen D, Tashman K, Palmer D S, Neale B, Roeder K, Bloemendal A, et al. A data harmonization pipeline to leverage external controls and boost power in GWAS. Hum Mol Genet. 2022; 31 (3): 481-489.
120. Watson D S. Interpretable machine learning for genomics. Hum Genet. 2022; 141 (9): 1499-1513.
121. Ma R, Cai TT, Li H. Global and Simultaneous Hypothesis Testing for High-Dimensional Logistic Regression Models. J Am Stat Assoc. 2021; 116 (534): 984-998.
122. Fu G-H, Wu Y-J, Zong M-J, Yi L-Z. Feature selection and classification by minimizing overlap degree for class-imbalanced data in metabolomics. Chemom Intell Lab Syst. 2020; 196:103906.
123. Rubin D B. Multiple imputation for nonresponse in surveys, vol. 81. John Wiley & Sons; 2004.
124. Guthrie W. et al. The earlier the better: an RCT of treatment timing effects for toddlers on the autism spectrum, Autism, 2023; https://doi.org/10.1177/13623613231159153

1. A method for providing early autism spectrum disorder (ASD) intervention to a newborn comprising the steps of:
a. identifying the newborn as having ASD or as being at risk of developing ASD by:
i. performing or having performed an ASD association assay on a sample from the newborn or a mother of the newborn to identify if the newborn has ASD or is at risk of developing ASD;
ii. identifying the newborn as having ASD or being at risk for developing ASD when the ASD association assay indicates that there is a dysregulation of the newborn's metabolomic profile; and
b. providing an early ASD intervention to the newborn so-identified.

2. The method of claim 1, wherein the newborn's metabolomic profile is compared to a control metabolomic profile.

3. The method of claim 1, wherein the newborn's dysregulated metabolomic profile is consistent with inflammation.

4. The method of claim 3, wherein the newborn is a male and the sample is blood or blood product from the mother at mid-gestation and wherein a decreased or lower level of the compound 17-hydroxy-4,7,10,13,15,19-docosahexaenoic acid or chemical cluster hydroxy eicosapentaenoic acid (HEPE) as compared to a control level identifies the newborn as having a dysregulated metabolomic profile consistent with inflammation and as having ASD or at risk for developing ASD.

5. The method of claim 3, wherein the newborn is a female and the sample is blood from the mother at mid-gestation and wherein an increased or higher ratio of arachidonic acid (AA)/eicosapentaenoic acid (EPA) as compared to a control level identifies the newborn as having a dysregulated metabolomic profile consistent with inflammation and as having ASD or at risk for developing ASD.

6. The method of claim 3, wherein the sample is cord blood and wherein an increased or higher level of the compound arachidonic acid (AA) or ratio of arachidonic acid (AA)/docosahexaenoic acid (DHA) as compared to a control level identifies the newborn as having a dysregulated metabolomic profile consistent with inflammation and as having ASD or at risk for developing ASD.

7. The method of claim 3, wherein the newborn is a male and the sample is cord blood and wherein a decreased or lower level of chemical cluster epoxyeicosatrienoic acid (EpETrE) as compared to a control level identifies the newborn as having a dysregulated metabolomic profile consistent with inflammation and as having ASD or at risk for developing ASD.

8. The method of claim 1, wherein the newborn's dysregulated metabolomic profile is consistent with disrupted membrane integrity.

9. The method of claim 8, wherein the sample is blood or a blood product from the mother at mid-gestation and wherein a decreased or lower level of chemical cluster phosphatidylcholines (PC) as compared to a control level, identifies the newborn as having a dysregulated metabolomic profile consistent with disrupted membrane integrity and as having ASD or at risk for developing ASD.

10. The method of claim 8, wherein the newborn is a female and the sample is blood or blood product from the mother at mid-gestation and wherein a decreased or lower level of compounds phosphatidylcholines (PC) or phosphatidylethanolamines (PE) or chemical cluster PE as compared to a control level, or an increased or higher level of chemical clusters PC-ether or PC ether-vlc as compared to a control level, identifies the newborn as having a dysregulated metabolomic profile consistent with disrupted membrane integrity and as having ASD or at risk for developing ASD.

11. The method of claim 8, wherein the sample is cord blood and wherein an increased or higher level of chemical cluster ceramide as compared to a control level identifies the newborn as having a dysregulated metabolomic profile consistent with disrupted membrane integrity and as having ASD or at risk for developing ASD.

12. The method of claim 8, wherein the newborn is a male and the sample is cord blood and wherein a decreased or lower level of chemical cluster phosphatidylcholines (PC) as compared to a control level, or an increased or higher level of chemical clusters carnitine, long chain ceramides or unsaturated ceramides as compared to a control level identifies the newborn as having a dysregulated metabolomic profile consistent with disrupted membrane integrity and as having ASD or at risk for developing ASD.

13. The method of claim 8, wherein the newborn is a female and the sample is cord blood and wherein an increased or higher level of chemical cluster sphingomyelin as compared to a control level, identifies the newborn as having a dysregulated metabolomic profile consistent with disrupted membrane integrity and as having ASD or at risk for developing ASD.

14. The method of claim 1, wherein the newborn's dysregulated metabolomic profile is consistent with impaired neurotransmission and neurotoxicity.

15. The method of claim 14, wherein the sample is blood from the mother at mid-gestation and wherein an increased or higher ratio of Glu/Gln as compared to a control level, identifies the newborn as having a dysregulated metabolomic profile consistent with impaired neurotransmission and neurotoxicity and as having ASD or at risk for developing ASD.

16. The method of claim 14, wherein the newborn is a female and the sample is blood from the mother at mid-gestation and wherein an increased or higher level of compound Glu as compared to a control level, identifies the newborn as having a dysregulated metabolomic profile consistent with impaired neurotransmission and neurotoxicity and as having ASD or at risk for developing ASD.

17. The method of claim 14, wherein the newborn is a male and the sample is blood from the mother at mid-gestation and wherein a decreased or lower level of compound Gln as compared to a control level, identifies the newborn as having a dysregulated metabolomic profile consistent with impaired neurotransmission and neurotoxicity and as having ASD or at risk for developing ASD.

18. The method of claim 14, wherein the newborn is a male and the sample is cord blood and wherein a decreased or lower level of Gln as compared to a control level, or an increased or higher ratio of Glu/Gln as compared to a control level, or chemical clusters long chain ceramides or unsaturated ceramides, identifies the newborn as having a dysregulated metabolomic profile consistent with impaired neurotransmission and neurotoxicity and as having ASD or at risk for developing ASD.

19. A method for providing early autism spectrum disorder (ASD) intervention to a newborn comprising the steps of:
a. identifying that the newborn has ASD or is at risk of developing ASD by:
i. performing or having performed an ASD association assay on a sample from a mother of the newborn to identify if the newborn has ASD or is at risk of developing ASD, wherein the sample is cord blood;
ii. identifying the newborn as having ASD or being at risk for developing ASD when there is high number of dysregulated chemical clusters in said sample as compared to a control number;
b. providing early ASD intervention to the newborn to the newborn so-identified.

20. A method for providing early autism spectrum disorder (ASD) intervention to a female newborn comprising the steps of:
a. identifying that the newborn has ASD or is at risk of developing ASD with increased impaired cognitive development by:
i. performing or having performed an ASD association assay on a sample to identify if the newborn has ASD or is at risk of developing ASD with increased impaired cognitive development, wherein the sample is blood from the mother at mid-gestation;
ii. identifying the newborn as having ASD or being at risk for developing ASD with increased impaired cognitive development when there is an imbalance of ether/non-ether phospholipids in said sample; and
b. providing early ASD intervention to the newborn identified with autism spectrum disorder (ASD) with increased impaired cognitive development;
or
a method for providing early intervention to newborn with autism spectrum disorder (ASD) comprising the steps of:
a. identifying that the newborn has ASD or is at risk of developing ASD by:
i. performing or having performed an ASD association assay on a sample to identify if the newborn has ASD or is at risk of developing ASD;
ii. identifying the newborn as having ASD or being at risk for developing ASD when there is a dysregulation of the newborn's metabolomic profile, wherein the metabolomic profile is determined using one or more of the metabolites listed in Table 7; and
b. providing early ASD intervention to the newborn.
